(19) United States
(12) Patent Application Publication
(10) Pub. No.: US 2020/0316360 A1
(43) Pub. Date: Oct. 8, 2020

(54) MEDICAL VESSELS WITH DYNAMIC FLUID TRANSFER LOCK MECHANISM

(71) Applicant: Mikael NAYGAUZ, Forrest Hills, NY (US)

(72) Inventor: Mikael NAYGAUZ, Forrest Hills, NY (US)

(21) Appl. No.: 16/423,120

(22) Filed: May 27, 2019

Related U.S. Application Data
(60) Provisional application No. 62/828,473, filed on Apr. 3, 2019.
(60) Provisional application No. 62/838,944, filed on Apr. 26, 2019.
(60) Provisional application No. 62/851,454, filed on May 22, 2019.

(51) Int. Cl. A61M 39/10; A61M 5/145; A61J 1/10; A61J 1/20

(52) U.S. Cl. CPC A61M 39/1011; A61M 5/145; A61J 1/2096; A61J 1/2055; A61J 1/2089; A61J 1/10

(57) ABSTRACT

The present invention relates, in some embodiments thereof, to devices, systems and methods for establishing a fluid passageway between vessels. In some embodiments, the invention provides a system that comprises a first vessel; a second vessel configured to be coupled to the first vessel; and a plunger locking mechanism disposed on the first vessel and configured to lock a plunger thereof and prevent the plunger from expelling content of the first vessel prior to an engagement with the second vessel, wherein the first and/or second vessel(s) comprise(s) an unlock mechanism configured to unlock the lock mechanism of the first vessel, and wherein at the time of or following the engagement between the first vessel and the second vessel, the unlock mechanism unlocks the lock mechanism of the plunger, allowing movement thereof and thereby a fluid transfer from the first vessel to the second vessel.

### CROSS REFERENCE TO RELATED APPLICATIONS

The present application claims priority to U.S. Provisional Patent Application Ser. No. 62/828,473, entitled SYSTEM, CONTAINER, AND METHOD FOR UNLOCKING PLUNGERS OF MODULARLY ASSEMBLED MEDICAMENT CONTAINERS and filed on Apr. 3, 2019; U.S. Provisional Patent Application Ser. No. 62/838,944 entitled SYSTEMS, CONTAINERS, AND METHODS HAVING PLUNGER LOCKING-UNLOCKING MECHANISMS and filed on Apr. 26, 2019; and U.S. Provisional Patent Application Ser. No. 62/851,454 entitled PLUNGER AND NEEDLE LOCKING AND UNLOCKING SYSTEMS and filed on May 22, 2019, the entire contents of which are expressly incorporated herein by reference.

All of the following United States Patent Applications are hereby incorporated herein by reference in their entireties: Ser. Nos. 16/100,840; 16/100,860; 16/100,874, 16/100,903; 16/169,695; 16/100,964; 16/100,969; 16/100,976; 16/100,981; 16/358,434; and 16/358,389.

### FIELD OF THE INVENTION

The present invention relates, in some embodiments thereof, to devices, systems and methods for establishing a fluid passageway between vessels. More particularly, the present invention relates, in one or more embodiments, to systems, devices, and methods for the prevention of spillage of beneficial substances from containers and/or medical devices.

### BACKGROUND OF THE INVENTION

The medicinal practice routinely involves administration of medical substances, such as, medicaments, fluids, nutrition substances and the alike, to patients. The preparation and/or administration of such medicinal substances typically involves one or more transfers of those substances between vessels (such as, vial, syringes, infusion lines, etc.).

Additionally, beneficial substances used in medical settings such as medicaments, nutritional substances, and the alike are provided in standard commercially available amounts. This poses a disadvantage for preparing tailored and/or customized final dosage amounts for patients. The preparation of customized amounts of intracorporeally administered beneficial substances requires the manual manipulation of the beneficial substances typically by healthcare personnel. Manual manipulation involves drawing out from vials using syringes and needles of amounts of beneficial substances, measuring visually the amount of the beneficial substances that has been drawn into a syringe, and injecting the amount of the beneficial substance from a syringe into a second container, typically a bag or a bottle. The preparation of customized amount of beneficial substances typically involves using less or more than the amount provided in one commercially available container. Customized dosages particularly of chemotherapeutic agents, are typically based on a patient's weight, height, and age which differs from patient to patient. Therefore, the preparation of customized final amounts of beneficial substances require the manual manipulation and measurement of beneficial substances by healthcare personnel.

Another important problem that exists when intermixing beneficial substances relates to the premature and/or accidental expulsion/spillage of a beneficial substance out of a container/device prior establishment of a fluid communication between containers/devices. For example, a pharmacist when mixing an injectable medicament may draw out a dose of the medicament from a vial into a syringe. The pharmacist may then accidentally push the plunger of the syringe before the syringe is engaged/connected to a second container such as a bag/bottle which would result in the expulsion/spillage of the medicament out of the syringe and into the environment and exposure of the pharmacist to the medicament. This is particularly dangerous if the medicament is a toxic substance such as a chemotherapeutic medicament which would result in the pharmacist being exposed to a toxic substance.

Yet another problem is associated with contamination of medical substances when intermixing beneficial substances. Atypically the action of fluid transfer exposes connection interfaces/surfaces of medical vessels to the environment and accordingly the medical substances themselves to contaminants present in ambient air particles (e.g., bacteria, viruses, funguses, spores, pyrogens). In addition, connection interfaces/surfaces of medical substances are further prone to contaminations due to physical contact of the interfaces/surfaces, for example, with nonsterile gloves, or devices. Such contaminations are a major problem in the healthcare setting since those, once invading within medicinal substances, may pose substantial danger to patients.

Taken together, the above described systems and methods are either cumbersome and expensive or inefficient in addressing the problem of intermixing customized amounts of beneficial substances while preventing the premature/accidental expulsion/spillage of the beneficial substances prior to establishment of a fluidic communication between containers/devices. Thus, there is a long felt and unmet need for systems, devices and/or methods that afford the safe transfer of medical substances between containers, and/or between containers and medicinal devices, while preventing the spillage of medical substances and exposure of healthcare personnel to toxic substances.

### SUMMARY OF THE INVENTION

Unless otherwise defined, all technical or/and scientific terms used herein have the same meaning as commonly understood by one of ordinary skill in the art to which the invention pertains. Although methods and materials similar or equivalent to those described herein can be used in the practice or testing of embodiments of the invention, exemplary methods or/and materials are described below. In case of conflict, the patent specification, including definitions, will control. In addition, the materials, methods, and examples are illustrative only and are not intended to be necessarily limiting.

The present invention pertains to devices, systems and methods for establishing a fluid communication between vessels in a secure manner and while allowing a fluidic transfer when vessels attach to each other. It is an object of the present invention, in some embodiments, to provide systems, devices, and methods of preventing spillage of beneficial substances/medicaments prior to engagement of vessels.

Thus, the invention provides in a first aspect a system for the engagement and establishment of fluid passageway between vessels, the system comprising:
a first vessel having a plunger;
a second vessel configured to be coupled to the first vessel; and
a plunger locking mechanism disposed on the first vessel and configured to lock the plunger thereof and prevent the plunger from expelling the content of the first vessel prior to an engagement with the second vessel, wherein the first and/or second vessel(s) comprise an unlocking mechanism configured to unlock the lock mechanism of the first vessel, and wherein at the time of or following the engagement between the first vessel and the second vessel, the plunger unlocking mechanism unlocks the lock mechanism of the plunger allowing movement thereof and thereby a fluid transfer from the first vessel into the second vessel.

In one or more embodiments, the lock mechanism is selected from a group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, and a combination thereof.

In one or more embodiments, the unlock mechanism is selected from the group consisting of a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, a hole, a dent, a socket, a channel, an elongated arm, a latch, a lock, a slot, and a combination thereof.

In one or more embodiments, the lock mechanism is moveable between a locked position wherein the lock mechanism protrudes from a side wall of the first vessel such to disrupt movement forward of the plunger and an unlocked retracted position wherein the plunger is capable of moving forward thereby expelling the substance into the first vessel.

In one or more embodiments, the lock mechanism is pivotally connected to a side wall of the first vessel and moveable about the pivot.

In one or more embodiments, the lock mechanism is connected to a wall of the first vessel via a hinge mechanism.

In one or more embodiments, the lock mechanism is spring loaded and/or attached to a spring, allowing planar movement thereof.

In one or more embodiments, the lock mechanism includes a retractable ball which can protrude into an inner lumen of the vessel thereby lock the plunger or retract to a socket in a wall of the first and/or second vessel, thereby unlock the plunger.

In one or more embodiments, the lock mechanism includes a magnetic ball which can protrude into an inner lumen of the vessel thereby lock the plunger or retract to a magnetic socket in a side wall of the first and/or second vessel, thereby unlock the plunger.

In one or more embodiments, the unlock mechanism includes an elongated arm disposed in the second vessel and configured to unlock the lock mechanisms.

In one or more embodiments, the system further comprises an engagement mechanism to connect the first vessel to the second vessel. In one or more embodiments, the engagement mechanism is selected from a thread, a luer, a ratchet teeth mechanism, a retention member, an adhesive mechanism, a clamping member and combinations thereof.

In one or more embodiments, the engagement mechanism disposed in an external position of the vessel. In one or more embodiments, the engagement mechanism disposed in an internal position of the vessel.

In one or more embodiments, the vessel is selected from the group consisting of a bag, a bottle, a syringe, an infusion line, a connector, a filter, a manifold, a bag port, a bottle port, a vial port, a cartridge and a combination thereof.

In one or more embodiments, the first and/or second vessel include(s) a port having peripheral walls enclosing a cavity, the cavity configured to receive or engage with a vessel, or a portion thereof. In one or more embodiments, the lock mechanism and/or the unlocking mechanism disposed on an internal and/or external position of the port. In one or more embodiments, the first and/or second vessel includes a port having peripheral walls enclosing a cavity, the cavity configured to receive or engage with a vessel, or a portion thereof, wherein the engagement mechanism disposed on an internal and/or external position of the port. In one or more embodiments, the first or second vessels configured to be coupled to a plurality of vessels. In one or more embodiments, the vessel having a plurality of cavities and/or a plurality of ports.

In one or more embodiments, the first vessel is configured to accommodate a fluid substance and the second vessel configured to receive a fluid substance from the first vessel.

In one or more embodiments, the first vessel is a cartridge. In one or more embodiments, the cartridge having a moveable wall. In one or more embodiments, the moveable wall is a plunger. In one or more embodiments, the first vessel is a plunger. In one or more embodiments, the first and/or second vessel includes a port having peripheral walls enclosing a cavity, the cavity is sized and shaped to accommodate the cartridge and/or syringe. In one or more embodiments, a plurality of cartridges are configured to be coupled to the second vessel.

In one or more embodiments, the first vessel includes a fluid transfer mechanism for transferring a fluid substance from the first vessel to the second vessel. In one or more embodiments, the fluid transfer mechanism includes a moveable plunger which when moved towards the first vessel's opening, allows the fluid transfer. In one or more embodiments, a hermetically sealed connection is formed between the first vessel and the second vessel following the vessels' engagement.

In one or more embodiments, the system further comprises a decontamination device configured to be coupled to the first vessel and second vessel and to allow the fluid passageway therebetween, the decontamination device further configured to decontaminate a surface of at least one of the first and second vessels.

In one or more embodiments, the system further comprises an engagement mechanism to connect the decontamination device to the first and/or second vessel. In one or more embodiments, the vessel and/or decontamination device includes a port having peripheral walls enclosing a cavity, the cavity configured to receive or engage with a vessel and/or decontamination device, or a portion thereof. In one or more embodiments, the decontamination device is pre-attached to the first or second vessels.

In one or more embodiments, the decontamination device is integrally attached to the first or second vessel.

In one or more embodiments, the decontamination device includes a wiping member configured to wipe off a surface of a vessel. In one or more embodiments, at least a portion of the first and/or second vessel is configured to move within the device and wherein the wiping member configured to remove contaminants from a surface of the first and/or second vessels at about the time of vessel's movement. In one or more embodiments, the wiping member is configured to move within the device such to allow decontamination of a surface of at least one of the first and second vessels at about the time of the wiping member's movement. In one or more embodiments, the decontamination device includes a sliding mechanism located on and/or in and/or attached to the wiping member and/or the decontamination device and/or the vessel and configured to allow for the movement of the wiping member and/or the vessel within the device. In one or more embodiments, the lock mechanism disposed on the first vessel and the unlock mechanism disposed on the decontamination device and/or the first vessel.

According to another aspect the present invention provides a method of establishing a fluid passageway between vessels, the method comprises the steps of:
providing a first vessel and a second vessel; wherein the first vessel comprises a plunger and a plunger lock mechanism and the first and/or second vessel(s) includes a plunger unlock mechanism; wherein the plunger lock mechanism configured to lock the plunger thus preventing the plunger from expelling a content within the first vessel; and wherein the plunger unlock mechanism configured to unlock the plunger lock mechanism;
connecting the first vessel to the second vessel;
unlocking the plunger lock mechanism; and
pushing a plunger of the first vessel towards the second vessel to thereby allow the fluid passageway between the vessels.

In one or more embodiments, an airtight seal is formed between the vessels following the step of connecting/engaging the vessels, allowing a hermetically sealed connection between the first vessel and the second vessel. In one or more embodiments, the step of connecting the vessels includes unlocking the lock mechanism. In one or more embodiments, the step of unlocking the lock mechanism occurs via a push motion, a twist motion, a turn motion or a combination thereof.

In one or more embodiments, the connection/engagement of the vessels occurs via a push motion, a twist motion, a turn motion or a combination thereof. In one or more embodiments, the connection/engagement of the vessels is selected from a twisting of a thread onto a complementary thread, attaching ratchet teeth to a complementary retention member, and adhering a surface of a first vessel to the surface of the second vessel via an adhesive, and combinations thereof.

In one or more embodiments, the plunger lock mechanism of the first vessel is selected from a pin, a hinge, an elongated arm, a channel, a dent, a latch, a spring, a magnet, and combinations thereof.

In one or more embodiments, the plunger unlock mechanism is selected from an elongated arm, a pin, a spring, a channel, a magnet, and combinations thereof.

In one or more embodiments, the method further comprises providing a decontamination device having a wiping member, the wiping member configured to decontaminate a surface of the first and/or second vessel(s).

In one or more embodiments, the method further comprises actuating the wiping member to move within the device, thereby decontaminating the surface of the first and/or second vessel.

In one or more embodiments, the method further comprises moving at least a portion of the first vessel and/or the second vessel within the device and via the wiping member, thereby decontaminating the surface of the first and/or second vessel.

In one or more embodiments, the vessel is selected from the group consisting of a bag, a bottle, a syringe, an infusion line, a connector, a filter, a manifold, a bag port, a bottle port, a vial port, a cartridge and a combination thereof.

In one or more embodiments, the method further comprises transferring a fluid substance between the vessels after engaging/connecting the vessels.

In one or more embodiments, the method further comprises transferring a fluid substance within the at least one vessel through the device after the surface of the at least one vessel has been decontaminated.

According to another aspect the present invention provides a system for establishing fluidic communication between containers, comprising:
a first container having a plunger and a plunger locking mechanism, the plunger locking mechanism configured to lock the plunger thus preventing the plunger from expelling contents of the first container out of the first container prior to engagement of the first container to a second container, the plunger locking mechanism further configured to be unlocked by the plunger unlocking mechanism of the second container;
a second container having a plunger unlocking mechanism, the plunger unlocking mechanism configured to unlock the plunger locking mechanism of the first container,
wherein upon engagement of the plunger unlocking mechanism of the second container and the plunger locking mechanism of the first container, the plunger locking mechanism is unlocked by the plunger unlocking mechanism thus allowing the plunger of the first container to move and expel the contents contained in the first container out of the first container and into the second container.

In one or more embodiments, the first container is a syringe. In one or more embodiments, the first container is a cartridge. In one or more embodiments, the first container is a vial. In one or more embodiments, the first container is made of a rigid material. In one or more embodiments, the first container is made of a glass material. In one or more embodiments, the second container is a bag. In one or more embodiments, the second container is a bottle. In one or more embodiments, the second container has a flexible wall, a rigid wall, or combinations thereof. In one or more embodiments, the unlocking mechanism comprising a pin, a retractable pin, a springe, and combinations thereof. In one or more embodiments, the unlocking mechanism comprising an elongated arm, a conduit, a vertical conduit, a conduit configured to allow entry of an elongated arm, and combinations thereof. In one or more embodiments, the unlocking mechanism comprising a magnet. In one or more embodiments, the unlocking mechanism comprising a pin, a channel, and combinations thereof. In one or more embodiments, the locking mechanism comprising at least one pin, at least one latch, at least one hinge, at least one spring, and combinations thereof.

According to another aspect the present invention provides a container having a plunger locking mechanism.

In one or more embodiments, the plunger locking mechanism is a locking mechanism. In one or more embodiments the plunger locking mechanism is selected from a pin, a latch, a spring, a magnet, and combinations thereof. In one or more embodiments, the container is a bag. In one or more embodiments, the container is a bottle. In one or more embodiments, the container is a syringe. In one or more embodiments, the container is a cartridge. In one or more embodiments, the container has a plunger. In one or more embodiments, the container is a vial. In one or more embodiments, the container is a bottle with a plunger. In one or more embodiments, the container has a moveable wall. In one or more embodiments, the plunger locking mechanism is configured to be unlocked by an unlocking mechanism disposed on a second container. In one or more embodiments, the second container is a bag or a bottle.

According to another aspect the present invention provides a container having a plunger unlocking mechanism. In one or more embodiments, the plunger unlocking mechanism is a pin. In one or more embodiments, the plunger unlocking mechanism is a retractable pin and spring mechanism. In one or more embodiments, the plunger unlocking mechanism is a magnet. In one or more embodiments, the plunger unlocking mechanism is disposed on the container. In one or more embodiments, the plunger unlocking mechanism is disposed in a cavity of a container. In one or more embodiments, the plunger unlocking mechanism is an elongated arm. In one or more embodiments, the elongated arm is disposed inside a cavity of the container. In one or more embodiments, the elongated arm is disposed on an exterior surface of the container. In one or more embodiments, the elongated arm is disposed inside the container. In one or more embodiments, the elongated arm is made of a rigid material. In one or more embodiments, the rigid material is a plastic material, a metal alloy, a glass material, and combinations thereof. In one or more embodiments, the plunger unlocking mechanism is configured to unlock a plunger locking mechanism of a second container. In one or more embodiment, the plunger unlocking mechanism is configured to unlock a plunger of a second container. In one or more embodiments, the container is a bag. In one or more embodiments, the container is a bottle. In one or more embodiments, the container is a manifold. In one or more embodiments, the manifold is configured to engage a plurality of syringe and/or cartridges. In one or more embodiments, the manifold is configured to engage a plurality of second containers. In one or more embodiments, the container is a connector. In one or more embodiments, the connector is configured to engage a plurality of second containers.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1D are front cut view illustrations demonstrating a system with a first vessel and a second vessel, the first vessel having a lock mechanism in the form of a moveable pin and an unlock mechanism in the form of a moveable pin, according to some embodiments of the invention.

FIGS. 2A-2D are front cut view illustrations demonstrating a system with a first vessel and a second vessel, the first vessel having a lock mechanism in the form of a moveable pin and an unlock mechanism in the form of a moveable pin disposed within a side wall of the first vessel, the system further comprising an unlock mechanism comprising a spring loaded pin disposed on a side wall of the second vessel, according to some embodiments of the invention.

FIGS. 3A-3D are front cut view illustrations demonstrating a system with a first vessel and a second vessel, the first vessel having a lock mechanism in the form of a retractable ball and an unlock mechanism in the form of a moveable pin, according to some embodiments of the invention.

FIGS. 4A-4D are front cut view illustrations demonstrating a system with a first vessel and a second vessel, the first vessel having a lock mechanism in the form of a retractable ball and an unlock mechanism in the form of a moveable pin disposed within a side wall of the first vessel, the unlock mechanism further comprises a spring loaded pin disposed on a side wall of the second vessel, according to some embodiments of the invention.

FIGS. 5A-5D are front cut view illustrations demonstrating a system with a first vessel and a second vessel, the first vessel having a lock mechanism in the form of a retractable ball and an unlock mechanism in the form of a moveable pin, the retractable ball resides in a dent within a plunger, according to some embodiments of the invention.

FIGS. 6A-6D are front cut view illustrations demonstrating a system with a first vessel, a second vessel, a dual lock mechanism having a combination a hinged pin and retractable ball and an unlocking mechanism in the form of pins, according to some embodiments of the invention.

FIGS. 7A-7C are front cut view illustrations demonstrating a system with a first vessel and a second vessel, the first vessel having a lock mechanism in the form of spring-loaded latch and an unlock mechanism in the form of a moveable pin, according to some embodiments of the invention.

FIGS. 8A-8D are front cut view illustrations demonstrating a system with a first vessel, a second vessel, a dual lock mechanism having retractable balls and unlocking mechanisms in the form of pins, according to some embodiments of the invention.

FIGS. 9A-9D are front cut view illustrations demonstrating a system with a first vessel, a second vessel, a dual lock mechanism having retractable balls and unlock mechanisms in the form of moveable pins disposed within a side wall of the first vessel and spring loaded pins disposed on the side walls of the second vessel, according to some embodiments of the invention.

FIGS. 10A-10D are front cut view illustrations demonstrating a system with a first vessel and a second vessel, the first vessel having a lock mechanism in the form of a magnetic ball and the second vessel having an unlock mechanism in the form of an elongated arm, according to some embodiments of the invention.

FIGS. 11A-11B are front cut view illustrations demonstrating a system with a first vessel having a syringe-like form, a second vessel for accommodating the first vessel, and a lock and unlock mechanisms having magnets, according to some embodiments of the invention.

FIGS. 12A-12B are front cut view illustrations demonstrating a system with a first vessel having a bottle-like form, a second vessel for accommodating the first vessel, and a lock and unlock mechanisms having magnets, according to some embodiments of the invention.

FIGS. 13A-13B are front cut view illustrations demonstrating a system with a first vessel having a cartridge-like form, a second vessel for accommodating the first vessel, and a lock and unlock mechanisms having magnets, according to some embodiments of the invention.

FIGS. 14A-14C are front cut view illustrations demonstrating a cartridge with a dual lock-unlock mechanisms, with hinged pins, according to some embodiments of the invention.

FIGS. 15A-15C are front cut view illustrations demonstrating a cartridge with a lock-unlock mechanisms, with hinged pins, the hinged pins of the unlock mechanism have a hook-like form, according to some embodiments of the invention.

FIGS. 16A-16D are front cut view illustrations demonstrating a system with a first vessel and a second vessel, the first vessel having a lock mechanism in the form of a moveable pin and an unlock mechanism in the form of a hook-like pin and the second vessel having an elongated arm, according to some embodiments of the invention.

FIG. 17 is a front cut view illustration demonstrating a system with a plurality of first vessels that can be engaged with a second vessel, the first vessels include a lock-unlock mechanism in the forms of moveable/hinged pins, according to some embodiments of the invention.

FIG. 18 is a front cut view illustration demonstrating a system with a plurality of first vessels in the form of syringes that can be engaged with a second vessel, the first vessels include a lock-unlock mechanism in the forms of moveable/hinged pins, according to some embodiments of the invention.

FIGS. 19A-19D are front cut view illustrations demonstrating a system with a first vessel in the form of a syringe, a second vessel, and a decontamination device, the first vessel having a lock mechanism in the form of a magnetic ball and the decontamination device has an unlock mechanism which magnetically retracts the lock mechanism, according to some embodiments of the invention.

FIGS. 20A-20B are front cut view illustrations demonstrating a system with a first vessel in the form of a cartridge, a second vessel, and a decontamination device, the first vessel having a lock mechanism in the form of a magnetic ball and the decontamination device has an unlock mechanism which magnetically retracts the unlock mechanism, according to some embodiments of the invention.

FIG. 21 is a front cut view illustration demonstrating a system with first vessels, second vessels and a decontamination device in the form of a connector, the vessels include a lock mechanism and the decontamination device includes an unlock mechanism, according to some embodiments of the invention.

FIG. 22 is a front cut view illustration demonstrating a system with first vessels, second vessels and a decontamination device in the form of a connector, the vessels include a hinged pin lock mechanism and the decontamination device includes an unlock mechanism, according to some embodiments of the invention.

FIG. 23 is a front cut view illustration demonstrating a system with first vessels, second vessels and a decontamination device in the form of a connector, the vessels include a hinged pin lock mechanism and a hook-like hinged pin unlock mechanism, the decontamination device includes an unlock mechanism in the form of an elongated arm, according to some embodiments of the invention.

FIG. 24 is a front cut view illustration demonstrating a system with first vessels, second vessels and a decontamination device in the form of a connector, the vessels include a retractable ball lock mechanism and a hinged/moveable pin unlock mechanism, according to some embodiments of the invention.

FIGS. 25A-25B are front cut view illustration demonstrating a system with a first vessel, a second vessel and a decontamination device, wherein at least the first vessel is moveable within the device, the first vessel includes a lock mechanism in the form of a retractable ball and an unlock mechanism having a moveable pin, according to some embodiments of the invention.

FIGS. 26A-26B are front cut view illustrations demonstrating a system with a first vessel, a second vessel and a decontamination device, wherein at least the first vessel is moveable within the device, the first vessel has a syringe-like form and includes a lock mechanism in the form of a retractable ball and an unlock mechanism having a moveable pin, according to some embodiments of the invention.

FIGS. 27A-27C are front cut view illustrations demonstrating a system with a first vessel, a second vessel and a decontamination device, wherein at least a portion of the first vessel is moveable within the decontamination device, the first vessel has a lock mechanism in the form of magnetically-retractable ball and the decontamination device includes an unlock mechanism in the form of an elongated arm, according to some embodiments of the invention.

FIGS. 28A-28B are front cut view illustrations demonstrating a system with a first vessel, a second vessel and a decontamination device, wherein at least a portion of the first vessel is moveable within the device, the first vessel has a lock and unlock mechanisms in the form of hinged pins, according to some embodiments of the invention.

FIGS. 29A-29B are front cut view illustrations demonstrating a system with a first vessel, a second vessel and a decontamination device, wherein at least a portion of the first vessel is moveable within the device, the first vessel has a lock mechanism in the form of a latch and an unlock mechanism in the form of a moveable/hinged pin, according to some embodiments of the invention.

FIGS. 30A-30B are front cut view illustrations demonstrating a system with a first vessel, a second vessel and a decontamination device, wherein at least a portion of the first vessel is moveable within the device, the first vessel has a lock mechanism in the form of a pin and an unlock mechanism in the form of a moveable/hinged pin disposed within a side wall of the first vessel, according to some embodiments of the invention.

FIGS. 31A-31B are front cut view illustrations demonstrating a system with a first vessel, a second vessel and a decontamination device, wherein at least a portion of the first vessel is moveable within the device, the first vessel has a cartridge-like form and a lock mechanism in the form of magnetically-retractable ball, the decontamination device includes an unlock mechanism in the form of an elongated arm, according to some embodiments of the invention.

FIG. 32 is a front cut view illustration demonstrating a system with first vessels, second vessels and a decontamination device in the form of a connector, wherein at least a portion of the first vessel is moveable within the decontamination device, and wherein the vessels include a lock and unlock mechanisms, according to some embodiments of the invention.

FIGS. 33A-33B are front isometric view illustrations demonstrating a system with a first vessel, a second vessel and a decontamination device, the first vessel is moveable within the decontamination device by rotation, and wherein the vessels include a lock and unlock mechanisms, according to some embodiments of the invention.

FIGS. 34A-34B are bottom isometric view illustrations demonstrating a system with a first vessel, a second vessel and a decontamination device, the first vessel is moveable within the decontamination device by rotation and has a syringe-like form, and wherein the vessels include a lock and unlock mechanisms, according to some embodiments of the invention.

FIGS. 34C-34E are top isometric view illustrations demonstrating a system with a first vessel, a second vessel and a decontamination device, the first vessel is moveable within the decontamination device by rotation and has a syringe-like form, and wherein the vessels include a lock and unlock mechanisms, according to some embodiments of the invention.

FIGS. 35A-35B are bottom isometric view illustrations demonstrating a system with a first vessel, a second vessel and a decontamination device, the first vessel is moveable within the decontamination device by rotation or pushing motion and has a finger protection unit, and wherein the vessels and/or decontamination device include a lock and unlock mechanism(s), according to some embodiments of the invention.

It should be appreciated that for simplicity and clarity of illustration, elements shown in the figures have not necessarily been drawn to scale. For example, the dimensions of some of the elements are exaggerated relative to each other for clarity. Further, where considered appropriate, reference numerals have been repeated among the figures to indicate corresponding elements.

### DETAILED DESCRIPTION OF THE INVENTION

It is understood that the invention is not limited to the particular methodology, devices, items or products etc., described herein, as these may vary as the skilled artisan will recognize. It is also to be understood that the terminology used herein is used for the purpose of describing particular embodiments only and is not intended to limit the scope of the invention. The following exemplary embodiments may be described in the context of exemplary locking mechanisms and unlocking mechanisms for ease of description and understanding. However, the invention is not limited to the specifically described products and methods and may be adapted to various applications without departing from the overall scope of the invention. All ranges disclosed herein include the endpoints. The use of the term “or” shall be construed to mean “and/or” unless the specific context indicates otherwise.

The present invention pertains to devices, methods and systems for fluid transfer between one or more vessels in a secure manner. The present invention pertains to systems, devices, and methods for preventing the premature spillage of beneficial substances/medicaments when transferred between containers/devices. The present invention pertains to systems, devices, and methods for preventing the premature spillage of beneficial substances/medicaments when transferred between containers/devices intended for use in a modular assembly format. The invention provides a solution to an unmet and long felt need in medical settings and allows for the locking of a plunger thus preventing expelling a medical substance, and unlocking of the plunger locking mechanism(s) at about the time, at the time, and/or after the engagement/coupling of at least two vessels/containers/devices. In one or more embodiments, the herein disclosed devices and systems pertain to a locking and unlocking mechanisms for securing and monitoring a fluid transfer between vessels. In one or more embodiments, the herein disclosed devices, systems, and/or methods pertain to plunger locking mechanism configured to secure/lock plungers of containers. In one or more embodiments, the herein disclosed devices, systems, and/or methods pertain to plunger unlocking mechanisms disposed on and/or in containers, the plunger unlocking mechanisms configured to unlock plungers of a second container. In one or more embodiments, the herein disclosed devices, systems, and/or methods pertain to systems comprising containers having plunger unlocking mechanisms and containers having plunger locking mechanisms, the plunger unlocking mechanisms configured to unlock plunger locking mechanism of a second container.

Thus, it is an object of the present invention, in some embodiments, to provide systems, devices, and methods for establishing fluidic communications between containers. It is an object of the present invention, in some embodiments, to provide vessels having a plunger locking mechanism(s). It is an object of the present invention, in some embodiments, to provide vessels having a plunger unlocking mechanism(s). It is an object of the present invention to provide vessels and/or decontamination devices having plunger unlocking mechanism(s) configured to unlock plunger locking mechanisms of other vessels. It is an object of the present invention to provide vessels having plunger locking mechanism(s) configured to be unlocked by plunger unlocking mechanisms of other vessel(s) and/or decontamination devices. It is an object of the present invention, in some embodiments, to provide systems, devices, and methods of preventing spillage of beneficial substances/medicaments prior to the engagement of at least two vessels.

Thus, in an aspect of the invention, there is provided a system for establishing a fluid passageway between vessels, the system comprises
a first vessel; and
a second vessel;
wherein the first vessel comprises a plunger locking mechanism configured to lock a plunger thereof and prevent the plunger from expelling the content of the first vessel prior to an engagement with the second vessel,
wherein the first and/or second vessel(s) comprise an unlocking mechanism configured to unlock the lock mechanism of the first vessel,
wherein at the time of or following the engagement between the first vessel and the second vessel, the plunger unlocking mechanism unlocks the lock mechanism of the plunger allowing movement thereof and thereby a fluid transfer from the first vessel into the second vessel.

In one or more embodiments, the herein disclosed devices and systems include a vessel with a plunger and a plunger lock mechanism that can lock the plunger, preventing premature transfer of the content from the vessel. Various forms of the lock mechanism are herein contemplated. Non-limiting examples of the locking mechanism include a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, a dent. The lock mechanism may be moveable between a locked position and an unlocked position. Optionally, the lock position includes extension of the lock mechanism into a hole/dent/socket/channel. Optionally, in the lock position, the lock mechanism is extended into the hole/dent/socket/channel and in the unlock position, the lock mechanism is retracted. The term “lock mechanism is interchangeable with the term “locking mechanism”.

In one or more embodiments, the herein disclosed devices and systems include an unlock mechanism that can unlock the lock mechanism of the plunger. In one or more embodiments, the unlock mechanism unlocks the lock mechanism at about the time of, at the time of, or following an engagement and/or coupling between a first vessel and a second vessel. Various forms of the unlock mechanism are herein contemplated. Non-limiting examples of the unlocking mechanism include a latch, a spring, a magnet, a hook, a tooth, a pin, a retractable pin, a ratchet tooth, a knob, a retractable knob, a ball, a retractable ball, a thread, a bangle, a locking key, a zip-tie, a moveable wall, a hole, a dent, a socket, a channel, an elongated arm, a latch, a lock, and a slot. The term “unlock mechanism is interchangeable with the term “unlocking mechanism”.

Various types, combinations and numbers of locking mechanisms may be provided in each of the herein disclosed systems, to ensure the locking action. For example, a combination of two locking mechanisms or more may be provided in each device/system/vessel.

Various types, combinations and numbers of unlocking mechanisms may be provided in each of the herein disclosed systems, to ensure the unlocking action. For example, a combination of two unlocking mechanisms or more may be provided in each device/system/vessel. In one or more embodiments, the locking mechanism is unlocked by the unlocking mechanism at the time and/or at about the time and/or after engagement of the first and second vessels. In one or more embodiments, the locking mechanism is unlocked by the unlocking mechanism after the engagement of the first and second vessels. In one or more embodiments, the locking and/or unlocking mechanism is disposed through a wall of the vessel. In one or more embodiments, the locking and/or unlocking mechanism is disposed on an interior wall of the vessel. In one or more embodiments, the locking and/or unlocking mechanism is disposed on an exterior wall of the vessel.

In one or more embodiments, the present invention provides a vessel having a moveable wall locking mechanism. In one or more embodiments, the present invention provides a vessel having a moveable wall and a moveable wall locking mechanism. In one or more embodiments, the present invention provides a vessel having a moveable wall unlocking mechanism. In one or more embodiments, the moveable wall may be a plunger. In one or more embodiments, the moveable wall may be made of a rubber material, a plastic material, a glass material, a metal alloy, and combinations thereof. In one or more embodiments, the moveable wall is configured to move within the vessel. In one or more embodiments, the moveable wall is configured to move within the vessel to expel the contents contained within the vessel out of the vessel.

Various forms of first and/or second vessels are contemplated. For example, the vessel may be a cartridge. In one or more embodiments, the cartridge includes protective side walls. In one or more embodiments, the moveable wall is a plunger. In one or more embodiments, the moveable wall is made of a rubber material. In one or more embodiments, the moveable wall is made of a plastic material. In one or more embodiments, the moveable wall is made of an elastomeric material. In one or more embodiments, the moveable wall is configured to move within the cartridge to expel the contents of the cartridge out of the cartridge. In one or more embodiments, at least one of the first and second vessels is a bag. In one or more embodiments, at least one of the first and second vessels is a bottle. In one or more embodiments, the first vessel is a bag and the second vessel is a syringe. In one or more embodiments, the first vessel is a syringe and the second vessel is a bag. In one or more embodiments, the first vessel is a bottle and the second vessel is a syringe. In one or more embodiments, the second vessel is a cartridge having a plunger. In one or more embodiments, the first vessel is a syringe and the second vessel is a bag. In one or more embodiments, the first vessel is a syringe and the second vessel is a bottle. In one or more embodiments, the first vessel is a cartridge and the second vessel is a bag. In one or more embodiments, the first vessel is a cartridge and the second vessel is a bottle. In one or more embodiments, the first vessel is a bottle and the second vessel is a bottle. In one or more embodiments, the first vessel is a vial and the second vessel is a bag.

In a further aspect, the present invention provides a method for establishing fluid communication between vessels, the method comprising:
providing a first vessel having a plunger and a plunger locking mechanism;
providing a second vessel having a plunger unlocking mechanism;
connecting the first vessel to the second vessel; and
pushing a plunger of the first vessels towards the second vessel to thereby establish a fluid passageway between the vessels, wherein the plunger locking mechanism configured to lock the plunger thus preventing the plunger from moving and expelling a substance from within the first vessel prior to the engagement between the first vessel and the second vessel, wherein the plunger unlocking mechanism configured to unlock the plunger locking mechanism of the first vessel; wherein upon the connection between the plunger unlocking mechanism and the plunger locking mechanism, the plunger locking mechanism is unlocked by the plunger unlocking mechanism thus allowing the plunger to move and expel the contents of the first vessel into the second vessel.

In one or more embodiments, the unlocking of the plunger locking mechanism is mediated via a pushing motion of the second vessel towards the first vessel. In one or more embodiments, the unlocking of the plunger locking mechanism is mediated via a pushing motion of the first vessel towards the second vessel. In one or more embodiments, the unlocking of the plunger locking mechanism is mediated via a pushing motion of the first and second vessels towards each other.

In one or more embodiments, the unlocking of the plunger locking mechanism is mediated via a twisting motion of the first vessel in relation to the second vessel. In one or more embodiments, the unlocking of the plunger locking mechanism is mediated via a twisting motion of the second vessel in relation to the first vessel. In one or more embodiments, the unlocking of the plunger locking mechanism is mediated via a twisting motion of the first and second vessels in relation to each other.

In one or more embodiments, the unlocking of the plunger locking mechanism is mediated via a turning motion of the first vessel in relation to the second vessel. In one or more embodiments, the unlocking of the plunger locking mechanism is mediated via a turning motion of the second vessel in relation to the first vessel. In one or more embodiments, the unlocking of the plunger locking mechanism is mediated via a turning motion of the first and second vessels in relation to each other.

In one or more embodiments, the unlocking of the plunger locking mechanism is mediated via a push and twist motion of the second vessel in relation to the first vessel. In one or more embodiments, the unlocking of the plunger locking mechanism is mediated via a push and twist motion of the first vessel in relation the second vessel. In one or more embodiments, the unlocking of the plunger locking mechanism is mediated via a push and twist motion of the first and second vessels in relation to each other.

In one or more embodiments, the unlocking of the plunger locking mechanism is mediated via a twist and push motion of the second vessel in relation to the first vessel. In one or more embodiments, the unlocking of the plunger locking mechanism is mediated via a twist and push motion of the first vessel in relation the second vessel. In one or more embodiments, the unlocking of the plunger locking mechanism is mediated via a twist and push motion of the first and second vessels in relation to each other.

In one or more embodiments, at least one of a push and twist motions moves the plunger locking mechanism from a locked position where the plunger is unable to move to an unlocked position where the plunger is can move forward.

In one embodiment, the present invention further provides devices, systems and methods configured to decontaminate one or more surfaces of a vessel(s). In one or more embodiments, the devices and systems of the invention allow a substantially decontaminated fluid transfer between the one or more vessel(s). The engagement and/or fluid transfer between the one or more vessels may accordingly be utilized via the decontamination devices which decontaminate the vessel(s) and also allow the fluid transfer. A locking mechanism and/or an unlocking mechanism may be disposed on the decontamination device, and one or both engaging vessels, thereby locking and unlocking a plunger.

In a further aspect, the present invention provides a system for decontaminating a surface of a vessel, the system comprising:
a first vessel having a plunger lock mechanism, the plunger lock mechanism configured to lock a plunger within the vessel thus preventing premature movement thereof;
a second vessel; and
a decontamination device having a first and second ports for connection to the first and second vessels, the decontamination device comprising a wiper/wiping member disposed therein the device and configured to remove contaminants from a surface of at least one of the first and second vessels, the decontamination device further comprises a plunger unlock mechanism configured to unlock a plunger lock mechanism of the first vessel,
wherein upon the engagement between the first vessel and the decontamination device, the lock mechanism is unlocked, allowing a fluid transfer between the first and second vessels via the decontamination device.

In one or more embodiments, the first vessel is a syringe. In one or more embodiments, the syringe has a plunger. In one or more embodiments, the first vessel is a cartridge. In one or more embodiments, the first vessel has a moveable wall. In one or more embodiments, the moveable wall is a plunger. In one or more embodiments, decontamination device has an actuator for effecting a wiping action of the wiping member. In one or more embodiments, the actuator is a handle. In one or more embodiments, the actuator is a tab.

In a further aspect, the present invention provides a method for establishing a fluid communication between vessels, the method comprises
providing a first vessel having a plunger lock mechanism, the plunger lock mechanism configured to lock a plunger within the vessel thus preventing movement of the plunger;
providing a second vessel; and
providing a decontamination device having a first and second ports for connection to the first and second vessels, the decontamination device comprising a wiper/wiping member disposed therein the device and configured to remove contaminants from a surface of at least one of the first and second vessels, the decontamination device further comprises a plunger unlock mechanism configured to unlock a plunger lock mechanism of the first vessel,
connecting the first vessel to the decontamination device;
wherein upon the engagement between the first vessel and the decontamination device, the plunger unlock mechanism of the decontamination device unlocks the plunger lock mechanism of the first vessel, thus allowing for the movement of the plunger of the first vessel to expel the contents of the vessel into the decontamination device and thereby establish a fluid communication between the first vessel and the second vessel.

In one or more embodiments, the unlocking of the plunger lock mechanism of the first vessel occurs due to a pushing motion of the decontamination device towards the first vessel. In one or more embodiments, the unlocking of the plunger lock mechanism of the vessel occurs due to a pushing motion of the vessel towards the decontamination device. In one or more embodiments, the unlocking of the plunger lock mechanism of the vessel occurs due to a pushing motion of the vessel and the decontamination device towards each other.

In one or more embodiments, the unlocking of the plunger lock mechanism of the vessel occurs due to a twisting/turning motion of the decontamination device relative to the vessel. In one or more embodiments, the unlocking of the plunger lock mechanism of the vessel occurs due to a twisting/turning motion of the vessel relative to the decontamination device. In one or more embodiments, the unlocking of the plunger lock mechanism of the vessel occurs due to a twisting/turning motion of the vessel and the decontamination device relative to each other.

In one or more embodiments, the unlocking of the plunger lock mechanism of the vessel occurs due to a push and twist motion of the decontamination device relative to the vessel. In one or more embodiments, the unlocking of the plunger lock mechanism of the vessel occurs due to a push and twist motion of the vessel relative to the decontamination device. In one or more embodiments, the unlocking of the plunger lock mechanism of the vessel occurs due to a push and twist motion of the vessel and the decontamination device towards each other.

In one or more embodiments, the unlocking of the plunger lock mechanism of the vessel occurs due to a twist and push motion of the decontamination device relative to the vessel. In one or more embodiments, the unlocking of the plunger lock mechanism of the vessel occurs due to a twist and push motion of the vessel relative to the decontamination device. In one or more embodiments, the unlocking of the plunger lock mechanism of the vessel occurs due to a twist and push motion of the vessel and the decontamination device towards each other.

Optionally, the decontamination device includes an actuator, disposed through a wall of the device, for manipulating the movement of the wiping member, and thereby decontaminating the surface of the first and/or second vessels.

Optionally, the locking mechanism may be disposed on the vessel and/or the unlocking mechanism on the decontamination device. Various positions of the locking/unlocking mechanism on the vessel and/or decontamination device are herein contemplated. The locking/unlocking mechanism may be disposed, without limitation, on a vessel's/device's body, port, aperture, wall, exterior, exterior wall, plunger and/or compartment. The locking mechanism may be disposed in an external and/or internal position of the vessel/device. Optionally, the decontamination device includes one or more compartments. Optionally, the decontamination device includes a first compartment thereof a connecting structure/port for receiving a vessel. The connecting structure/port may mediate a connection with a vessel, via an engagement mechanism. Various forms of connecting structures/ports are herein contemplated. In one or more embodiments, the connecting structure/port extends downwardly from a top inner wall of a first compartment. In one or more embodiments, the connecting structure/port includes extension bodies (e.g., an elongated arm), clamping members, a threaded structure, minimized built-in dent structures, elongated extensions, and a circular body.

The decontamination device of the invention includes a housing having side walls, a top wall and a bottom wall, and a wiping member disposed within the housing. The device configured to be coupled to at least two vessels and includes applicable connecting structures/ports or openings for receiving the vessels. The connecting structures/ports/openings may optionally be formed by vertical walls extending from the top and/or bottom walls, forming an inner lumen via which medical substances may be transferred following engagement between, and decontamination of the vessel surfaces. Other port configurations include ports/openings without any walls, tilted walls, etc. The ports may be flush, or surface mounted to the housing. Optionally, a locking mechanism and/or unlocking mechanism may be disposed in a port of the device, for example, in an internal position within vertical walls of the port. Optionally, a locking mechanism and/or unlocking mechanism may be disposed on a surface, and exterior surface, or an interior surface of a decontamination device and/or a vessel.

The wiping member is configured to decontaminate a vessel's surface, optionally by a movement of the wiping member across the housing and a surface of the vessel, or by movement of the surface of the vessel within the housing. The wiping member may be moveable or may be static.

In an embodiment of the invention, the wiping member is operable to move across and wipe off any contaminates present on the one or more surfaces of the vessels via an actuator extending through an opening of the housing. The actuator is configured to manipulate movement of the wiping member across the housing. In one or more embodiments, the wiping member is operable to move across and wipe off any contaminants present on the one or more surfaces of the vessels via an actuator extending through an opening in the side wall of the housing. Exemplary actuators include, without limitation, a handle, a tab, a button, a touch button, a lever, a gear, a spring, a mechanical actuator, an electric actuator, an air pressure actuator, and combinations thereof.

In one or more embodiments, the wiping member is operable to move across and wipe off any contaminates present on the one or more surfaces of the vessels without any actuator for mediating the wiping action. In accordance with this embodiment, a user may, for example, apply force onto the housing to thereby forcibly manipulate the wiping member to slide across the housing. In accordance with this embodiment, the housing may be flexible, so the user can manipulate the wiping member from the outside and by applying for example press forces onto the housing. In one or more embodiments, the wiping member is moveable from an initial position which precedes the wiping action to an end position which follows the wiping action of the wiping member.

Optionally, the wiping member is static within the housing and the decontamination of a vessel's surface is effective via movement of at least a portion of the vessel within the housing and via the wiping member. In accordance with this embodiment, the system optionally comprises a sliding mechanism positioned on at least one of the first vessel and the second vessel, and/or within/on the housing, the sliding mechanism configured to allow traveling there along of at least one of the first vessel and the second vessel, such that at least one of the first vessel and the second vessel is configured to move from a first position to a second position. The sliding mechanism may in one or more embodiments be a rail mechanism. The wiping member is configured to remove contaminants from a surface of at least one of the first vessel and the second vessel at about the time of movement of the medical vessels from the first position to the second position. In one or more embodiments, the medical vessel or medical vessel surface slides along the housing and passes through and/or under and/or over the wiping member under great friction, or under a friction sufficient to wipe off any contaminants from the surface of the vessel, such as dirt, bacteria and/or ambient air particles.

Various types of sliding mechanisms are contemplated as long as they can be connected or engage a vessel and afford traveling there along and allow decontamination of a surface of the vessel. The sliding mechanism may include a rail. The rail may be optionally made from a plastic or polymetric material, but a metallic material or glass material is also applicable. In one embodiment, the rail is manufactured from a plastic material. In one or more embodiments, the plastic material is rigid. In one or more embodiments the vessel surface is positioned on a surface of the rail. In one or more embodiments, the vessel surface is positioned between the rails of the rail. In one or more embodiments, the vessel surface is positioned between a portion of the rails. In one or more embodiments, the rail may be a circumferential rail. In one or more embodiments, the sliding mechanism may include a hinge/pivot mechanism. In one or more embodiments, the sliding mechanism provides for the travelling a vessel between a plurality of positions, such as from a second position to a third position, from a third position to a fourth position, and from a fourth position to a fifth position. In one or more embodiments, the sliding mechanism allows only unidirectional sliding. Alternatively, the sliding mechanism allows for the vessel to retract/return to a previous position.

In one or more embodiments, the wiping member is manufactured from or comprises a material including, but not limited to an elastic material, a rigid material, a flexible material, an expandable material, a polymer, and combinations thereof. In one or more embodiments, the wiping member is manufactured from or comprises an elastomeric material, such as, but not limited to a rubber material. In one or more embodiments, the wiping member is manufactured from or comprises a sponge or a sponge-like material. In one or more embodiments, the wiping member slides across the surface of the one or more vessel(s) under great friction, or under a friction sufficient to wipe off any contaminants from the surface, such as dirt or bacteria. In one or more embodiments, the vessel's surface slides underneath the wiping member under great friction, or under a friction sufficient to wipe off any contaminants from the surface, such as dirt or bacteria.

In one or more embodiments, the wiping member is covered, at least partially, by a sterilizing substance. As used herein the term “sterilizing substance” encompasses any substance that can eliminate or reduce the presence of microorganisms, air particles, or any of the alike. In one embodiment, the term “sterilizing substance” is interchangeable with the term “disinfecting substance”. In one or more embodiments, the sterilizing substance may be, but is not limited to, an antibacterial substance, an antiviral substance (e.g., an anti-HIV agent), an anti-tuberculin substance, an anti-fungal, and combinations thereof.

In one or more embodiments, the sterilizing substance may be a bactericidal substance, a virucidal substance, and/or a fungicidal substance. Exemplary sterilizing substances include, without limitation, alcohol, isopropyl alcohol, and hydrogen peroxide. In one or more embodiments, the sterilizing or disinfecting substance may be a fluid or a liquid. In one or more embodiments, the sterilizing or disinfecting substance may be a gas. In one or more embodiments, the gas may be a pressurized gas. In one or more embodiments, the sterilizing substance may be housed within the housing of the decontamination device. In one or more embodiments, at least one of the openings and/or ports of the decontamination device are sealed and/or covered thus preventing the sterilizing substance from escaping from inside the housing of the decontamination device. The seals and/or covers maintain the sterility of the decontamination device until about the time of use of the decontamination device. In one or more embodiments, the sterilizing substance is sodium hypochlorite. In one or more embodiments, the concentration of sodium hypochlorite is 0.55% or higher. In one or more embodiments, the concentration of sodium hypochlorite is below 0.55% concentration.

In one or more embodiments, the housing may be manufactured from various materials. Optionally, the housing is manufactured from an inert material, thus suitable for various types of pharmaceutical substances. The housing may be made from flexible or from rigid materials. Suitable materials include, without limitation a plastic, a glass, a rigid plastic, a flexible plastic, and combinations thereof. In one or more embodiments, the housing may be transparent. In one or more embodiments, the housing may be opaque.

Various sizes and shapes of the housing are applicable. For example, the housing may have an external most length, width, and/or height of less than about 3 inches. In one or more embodiments, the housing may have an external most length, width, and/or height of less than about 25 millimeters (mm). For example, less than about 20 mm, less than about 15 mm, or less than about 10 mm. The housing internal most length, width, and/or height may be less than about 10 mm. For example, less than about 4 mm, less than about 3 mm, or less than about 2 mm. The housing may include one or more compartments which may optionally be separated or divided by the wiping member or by any other means. In one or more embodiments, the housing includes two compartments, wherein a first compartment is kept sterile and a second compartment is configured to receive a vessel which may be contaminated. In one or more embodiments, the sterile compartment can receive a vessel following decontamination of the surface of the vessel. In one or more embodiments, the housing includes two compartments wherein a first includes a capping mechanism and the second is configured to receive a vessel. In one or more embodiments, the housing includes three, four, or five compartments. The vessel may move from a first position in which a vessel surface is located within the first compartment to a second position in which the vessel surface is located in the second compartment. Optionally, the housing includes a third compartment and the vessel surface is moveable from the second position to a third position. Optionally, the housing includes a fourth compartment, or a fifth compartment and the vessel surface is moveable from a third position to a fourth position and/or a fifth position.

In one or more embodiments, the housing is conveniently light presenting a weight of no more than about 100 grams (gr). For example, less than about 75 gr, less than about 50 mm gr, less than about 25 gr, less than about 10 gr, or less than about 5 gr. In one or more embodiments, the housing may be transparent allowing visualization of its interior, or opaque.

In one or more embodiments, the housing may be a closed chamber which forms a tight, optionally, airtight connection between the vessels. In one or more embodiments, when the ports or openings are connected to vessels or when the ports are covered by a cover or a seal, the housing may form an interior which is hermetically sealed/isolated from surrounding/environmental air. In one or more embodiments, the cover or the seal is frangible, breakable or rupturable. In one or more embodiments, the cover or the seal is pierceable by a piercing member such as a needle.

In one or more embodiments, the housing may optionally include one or more apertures, optionally covered by a filter, to thereby allow releasing air from the housing when actuating the wiping member. Optionally, the one or more apertures may be covered by a valve, to thereby allow releasing of air or pressure from within the housing when actuating the wiping member. In one or more embodiments, the valve may be a one-way valve, thereby allowing the release of air or pressure from within the housing when actuating the wiping member while preventing ambient or environmental air from entering the housing. In one or more embodiments, the filter or valve may be located in or on a wall of the housing's first, second or third compartment.

In one or more embodiments, the system or devices of the invention is provided when the housing is already connected to one or more medical vessels. In accordance with this embodiment, the one or more medical vessels may be integrally manufactured or assembled with the housing of the decontamination device and provided or distributed to consumers as such. In accordance with this embodiment, the system may be configured to allow a connection/locking/engagement to additional one or more vessels.

In one or more embodiments, the system includes two or more ports which are disposed one above the other, or which are longitudinally aligned with respect to each other. In accordance with this embodiment, the vessels connected to such ports are positioned aligned allowing a direct and aligned fluid passageway between the vessels. In one or more embodiments, the system includes two or more ports which are shifted from each other. Yet, in accordance with this embodiment, a fluid passageway is formed between vessels which is twisted or curved. In yet one or more embodiments, the housing contains a plurality of ports configured for connecting a plurality of vessels to the housing. The system or device is configured so that actuating the wiping member allows wiping the surfaces of all vessels connected to the housing in one action.

In one or more embodiments, the housing has an engagement mechanism configured or adapted to connect the at least one vessel to the housing. In one or more embodiments, the engagement mechanism may be reversible, allowing to detach the engagement between a vessel and a decontamination device. Various types of engagement mechanisms may be applicable and are contemplated. For example, the engagement mechanism is selected from, without limitation, a thread, a luer, a luer-lock, a luer-slip, snap-on, twist-on, a ratchet teeth mechanism, a retention member, a clamping rail, and an adhesive mechanism. The thread and the complementary thread may be selected from, without limitation, a luer, a smart-site mechanism and combinations thereof. In an embodiment, the engagement mechanism may be selected from a ratchet teeth mechanism, a clamping rail mechanism and/or an adhesive. In one or more embodiments, the herein disclosed invention allows transferring medical substances in a contaminant-free, or in a substantially contaminant-free manner.

In one or more embodiments, the herein disclosed invention affords an engagement of vessels in a contaminant-free, or in a substantially contaminant-free manner. In one or more embodiments, the herein disclosed invention affords an engagement of vessels in an air-free, or in a substantially air-free manner.

In one or more embodiments, the herein disclosed invention provides a fluidic passageway or communication between vessels in a contaminant-free, or in a substantially contaminant-free manner. In one or more embodiments, the herein disclosed invention provides a fluidic passageway or communication between vessels in an air-free, or in a substantially air-free manner.

In one or more embodiments, the herein disclosed invention, allows to substantially decrease the chances to introduce contaminants within a medical substance, when preparing or administering medical substances for administration to patients.

As used herein the term “substances” refers to various types of materials that should be kept sterile. The substances may be liquid, semi-solid, or gas. In one or more embodiments, the substances are “medical substances”. As used herein the term “medical substances” refers to and encompasses any of the various pharmaceutical drugs, fluids, nutritional products and the like. In an embodiment of the invention the term “medical substance” is interchangeable with the terms a “pharmaceutical substance”, “a beneficial substance” and “a medicament”.

As used herein the term “contaminant-free” is interchangeable with the term “sterile”, “disinfected”, and “decontaminated”. The term refers to substances that are free or substantially free of ambient air particles and/or pathogens and/or micro-organisms, and/or any of the alike. Typically, when less or no air is introduced within medical substances, the chances of contamination by pathogens, such as, bacteria, viruses, funguses, spores, pyrogens or the alike is completely abolished or significantly reduced.

As used herein, the term “substantially contaminant-free” means significantly less ambient air present when transferring medical substances with the herein disclosed vessels and systems, as compared to comparable conditions for transferring medical substances without the herein disclosed vessels and systems.

As used herein the term “ambient air particles” is interchangeable with the term “environmental air particles” and refers to air particles present in a non-filtered environment. The air particles may contain dirt, such as dust. Alternatively, or additionally, the air particles may contain pathogens or other microorganisms, or any of the alike.

As used herein the term “vessel” refers to any device utilized for containing, transferring or administering substances as herein disclosed. In one or more embodiments, the vessels may be used for containing medical substances. In an embodiment of the invention, the vessel is a medical vessel. In one or more embodiments, the term “medical vessel” is interchangeable with the term “pharmaceutical vessel”. In an embodiment of the invention, the vessel is a medical device. In an embodiment of the invention, the vessels are used for, and adapted to allow connection to another vessel. In an embodiment of the invention, the vessel is utilized for containing or housing a medical substance. Various types of vessels are contemplated. The vessel may be selected, without limitation, from a vial, a syringe, a bag, a chamber, a bottle, a container, a cartridge and the alike. In an embodiment of the invention, the term vessel further encompasses elements that can be used to connect between vessels. In accordance with this embodiment, the vessel may be selected, without limitation, from a connector, a connector having a plurality of openings, a syringe, an infusion line, a tubing, an infusion line, a spike, a syringe, a filter, a port and a manifold. In one or more embodiments, the first container is a bag. In one or more embodiments, the herein system include a first vessel and a second vessel. In one or more embodiments, the first vessel is a cartridge configured to fill the second vessel. In accordance with this embodiment, the cartridge includes a mechanism for allowing the fluid transfer. Suitable fluid transfer mechanisms may include, without limitation a plunger, and a moveable wall. Optionally, the cartridge may have various forms, such as a syringe, a vial, a bottle, a bag, etc. In one or more embodiments, the first vessel is a bottle. In one or more embodiments, the first vessel is a syringe and the second vessel is a bag, or vice versa. In one or more embodiments, the first vessel is a syringe and the second vessel is a bottle, or vice versa. In one or more embodiments, the first vessel is a cartridge with a moveable wall and the second vessel is a bag. In one or more embodiments, the first vessel is a cartridge with a plunger and the second vessel is a bag. In one or more embodiments, the first vessel is a cartridge with a moveable wall and the second vessel is a bottle. In one or more embodiments, the first vessel is a cartridge with a plunger and the second vessel is a bottle. In one or more embodiments, at least one of the first and second containers is a bag. In one or more embodiments, at least one of the first and second vessels is a bottle. In one or more embodiments, the vessel is a cartridge having a plunger and protective side walls.

As used herein the term “fluid communication” refers to two or more vessels in which substances may pass therethrough either directly or indirectly. The fluid communication may occur via a fluid passageway that allows for the flow/transfer of substances. In an embodiment of the invention, fluid passageway between vessels is established when any seals or covers of the ports of the herein disclosed systems and/or of the vessels are open. Optionally, the vessels and/or ports of the systems are provided with seals/covers and those seals/cover become open upon piercing by a piercing member or breaking/rupturing thereof. Optionally, the vessels and/or ports of the systems are provided with seals/covers and those seals/covers are opened upon uncovering or removing thereof by a user. Optionally, one or more of the seals/covers disclosed herein are fragile, allowing piercing thereof with a sharp instrument (i.e., a piercing member) or by the application of a sufficient force to break or rupture the seals/covers. The piercing member may optionally be a needle, for example, a hollowed needle, but other sharp elements are contemplated. In one or more embodiments, the seals/covers may be peelable.

In an embodiment of the invention, the herein disclosed devices, systems and methods allow fluid communication in a contaminant-free, or in a substantially contaminant-free manner. For example, between three or more, four or more, five or more or six or more vessels. In an embodiment of the invention, the herein disclosed devices, systems and methods allow fluid communication between three, four, five six, or seven vessels. For example, the devices, systems, and methods of the invention allow fluid communication between two bags, between two containers, between two bottles, between a syringe and a bag, between a syringe and a container, between a syringe and a vial, between a connector and a bag, between a container and a bag, between a vial and a bag, between a container and an infusion line or between a syringe and a connector. In an embodiment of the invention, the herein disclosed devices and systems are disposable. In an embodiment of the invention, the herein disclosed devices and systems are non-disposable.

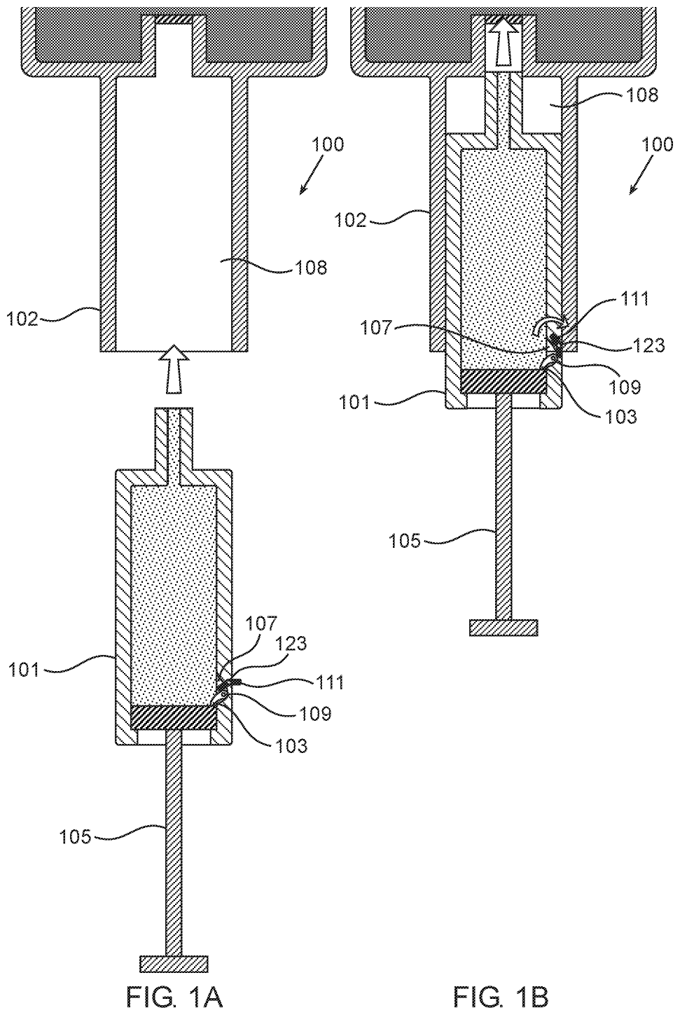

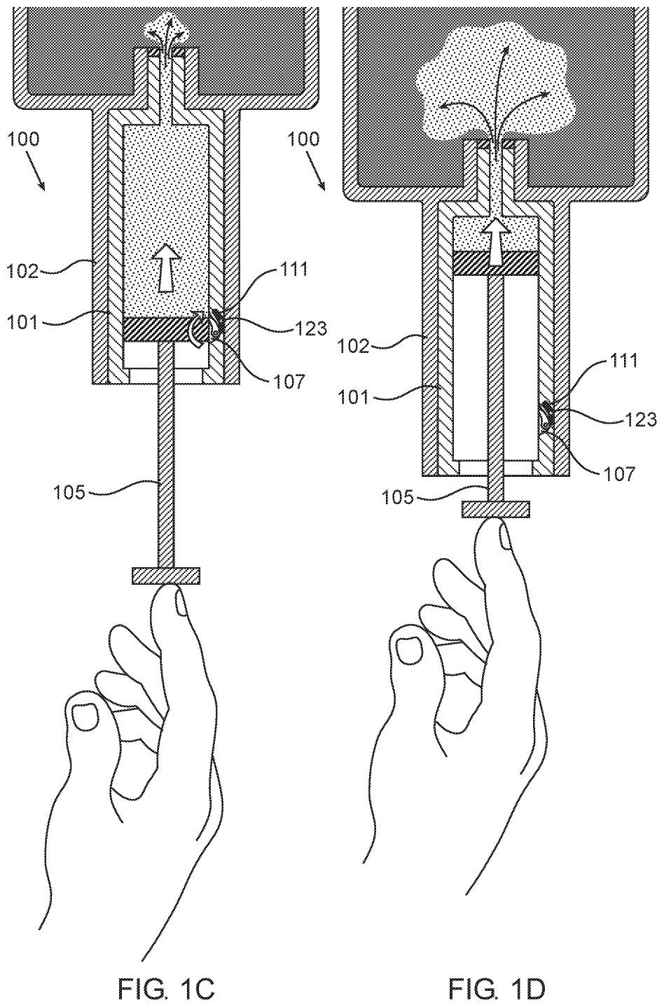

Referring now to the drawings, FIGS. 1A-1D illustrate a system 100 for establishment of a fluid communication between vessels. System 100 includes a first vessel, here syringe 101, and a second vessel 102 which is adapted to accommodate/receive syringe 101. Second vessel 102 may include a cavity 108 for accommodating therein syringe 101. As shown in FIGS. 1C-1D, once connected, a fluid passageway may be formed between the vessels 101 and 102. The fluid passageway may be effected via injection of a substance from vessel 101 to vessel 102 by pushing plunger 105. The system 100 includes a plunger locking mechanism 103 in order to monitor the fluid transfer and/or prevent a premature fluid transfer. Plunger locking mechanism 103 has a pin-like form, that prevents premature/accidental movement of the plunger 105 of syringe 101. The lock mechanism 103 is connected to an inner wall of the syringe 101 and protrudes therefrom, nevertheless, alternative positions of the lock mechanism 103 may be applicable and are contemplated. The lock mechanism 103 may extend from a socket 107 and protrude inwardly into a lumen of the syringe 101. Optionally, the lock mechanism 103 is attached to a wall in syringe 101 via a hinge 109 which allows movement of the lock mechanism 103 between a locked position and an unlocked retracted position. As herein illustrated, in the lock position (shown in FIGS. 1A-1B), lock mechanism 103 presses a horizontal wall of the plunger 105 and prevents the plunger 105 from being pushed upwardly and ejecting the medical substance. In the unlocked position (shown in FIGS. 1C-1D), the lock mechanism 103 is being pressed up against an inner side wall of the syringe 101. The lock mechanism 103 is retracted into a wall of the syringe, optionally into socket 107, allowing the plunger 105 to move forward and expel contents of the syringe 101. The lock mechanism 103 may be made of various materials, e.g., a rubber material, a metal material, a glass material, and/or any inert material that will not interact with a beneficial substances/medicament disposed inside the syringe 101. System 100 further includes an unlocking mechanism 111, having a pin-like form, for securing the lock position and mediating the unlocking of the lock mechanism 103 at about the time of/at the time of/following the engagement between vessels 101 and 102. Unlocking mechanism 111 may also have a hinge 123 that allows movement thereof. As illustrated in FIG. 1A, at the lock position, unlocking mechanism 111 maintains the lock mechanism 103 in the locking position by pressing it downwardly. The unlock mechanism 111 protrudes outwardly, to an external space, from a side wall of the syringe 101 such that upon the engagement between the first and second vessels 101 and 102, the protruding portion of unlocking mechanism 111 is pushed downwardly/retracted such to release lock mechanism 103 (FIG. 1B). A user can then push plunger 105 and eject/expel the beneficial substances/medicament from the first vessel 101 into the second vessel 102 (FIGS. 1C and 1D).

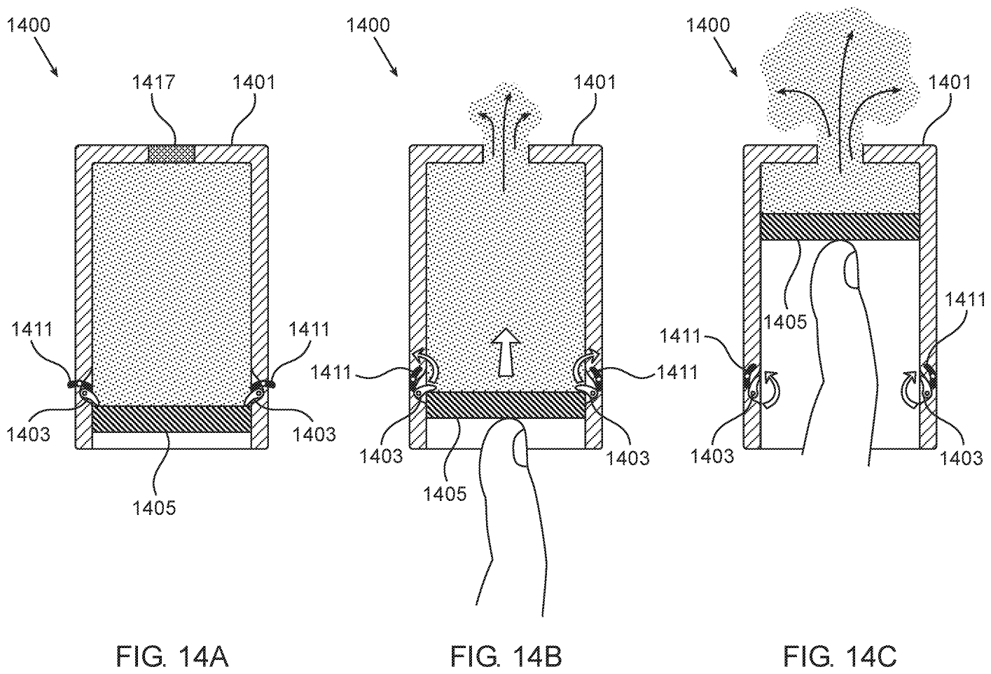

It is to be noted that similar lock-unlock mechanisms may be incorporated to various forms of vessels, such as cartridges, vials, bottles, etc. As shown in FIGS. 14A-14C, similar lock-unlock mechanisms, with hinged pins 1403 and 1411 are incorporated to cartridge 1401 which here includes a dual lock mechanism 1403 and a dual unlock mechanism 1411 disposed on opposing side walls of cartridge 1401. When unlock mechanisms 1411 unlock lock mechanisms 1403, the plunger 1405 can be released such that a user can eject/expel a medical substance out of cartridge 1401 and release thereof via aperture 1417.

As depicted herein above and in the following examples, various forms of the lock/unlock mechanisms are applicable and contemplated, for example, the lock/unlock mechanisms may include a pin (e.g., a retractable/rotatable pin), a ball (e.g., a retractable/rotatable ball), a clamp, a latch, an elongated arm, and combinations thereof.

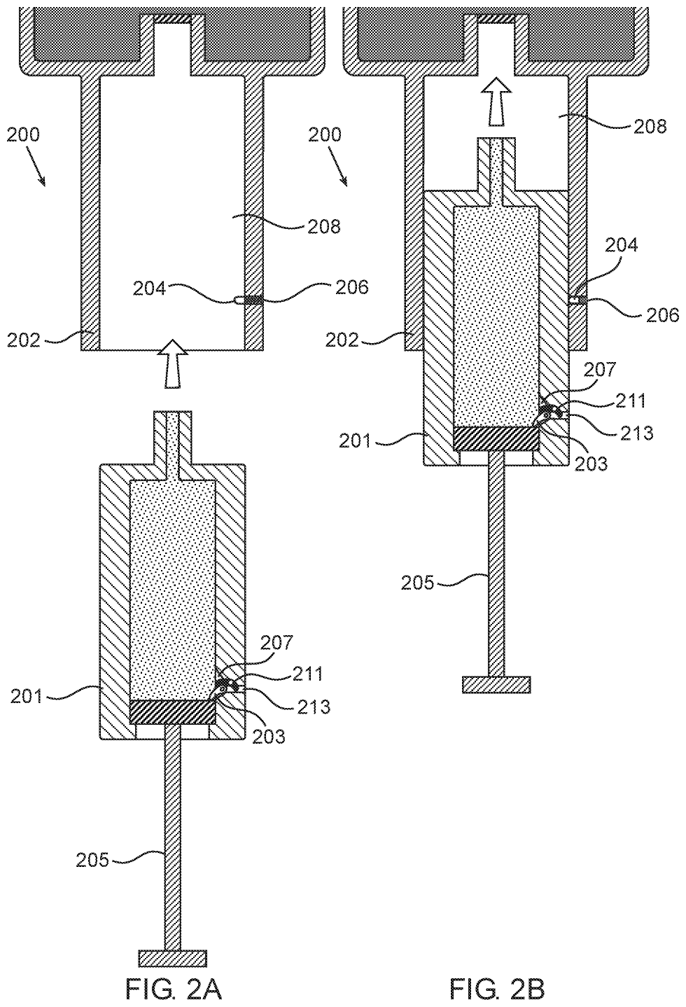

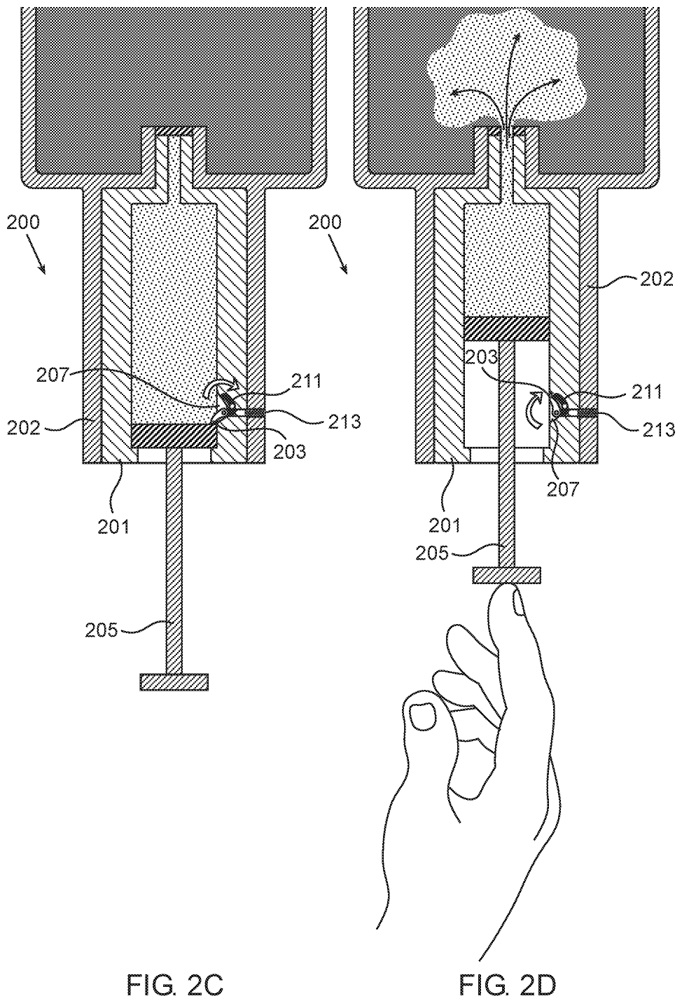

FIGS. 2A-2D illustrate yet another exemplary system 200 which includes a first and second medical vessels 201 and 202, for transferring a beneficial/medical substance therebetween. Similarly to system 100, the lock mechanism 203 and unlocking mechanism 211 are hingedly connected to a side wall of a syringe 201 with the exception that the unlocking mechanism 211 is disposed entirely within a side wall of syringe 201, optionally, within, connected to or extending from channel 213. The unlocking mechanism 211 blocks at least a portion of lock mechanism 203 and thereby locks locking mechanism 203, maintaining same at the lock position (FIGS. 2A-2B). The unlocking mechanism 211 includes a spring 206 loaded pin 204 disposed to protrude from a side wall of vessel 202. Upon entry of vessel 201 into vessel 202, pin 204 is firstly retracted and thereafter, following a final positioning of the syringe 201 in a cavity 208 of vessel 202, pin 204 can be expanded such to enter channel 213. The insertion of pin 204 into the channel 213 pushes the unlocking mechanism 211 upwardly, releasing locking mechanism 203. A user can then push plunger 205 upwardly and inject the beneficial substances/medicament into second vessel 202 (FIG. 2D).

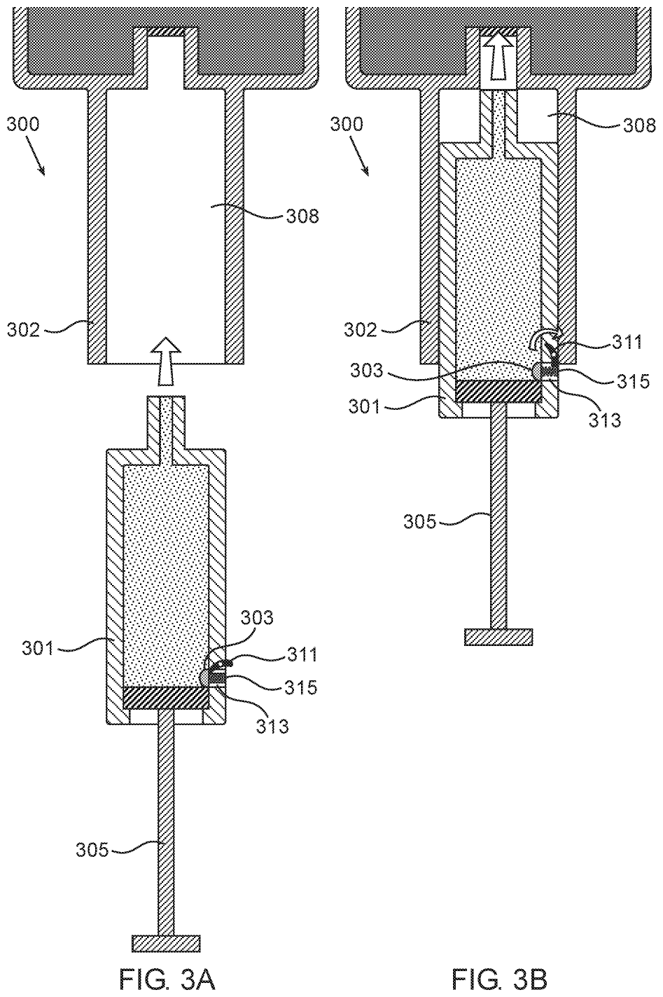

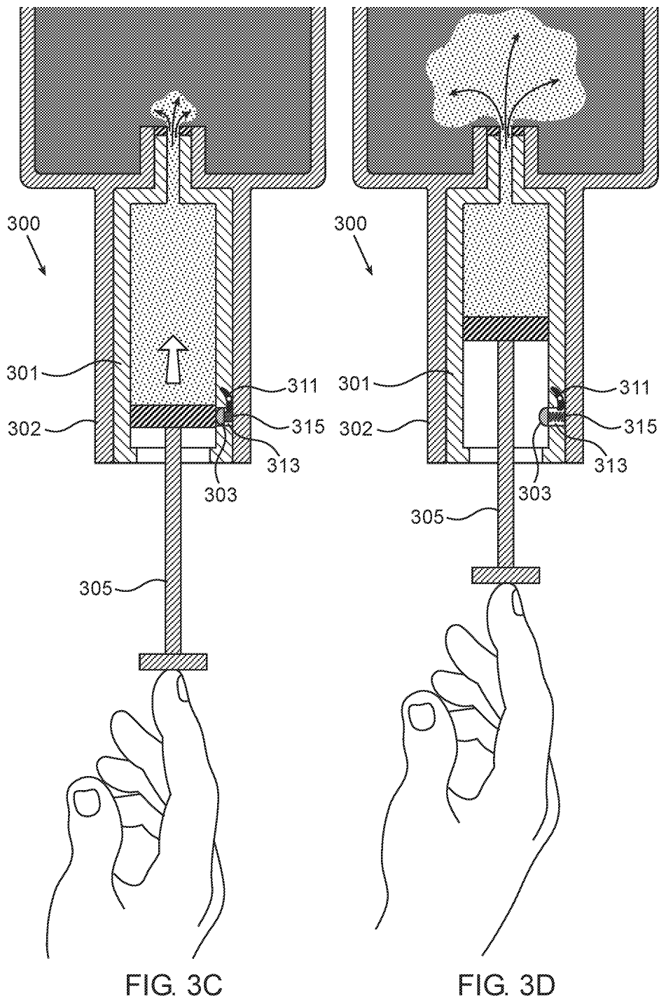

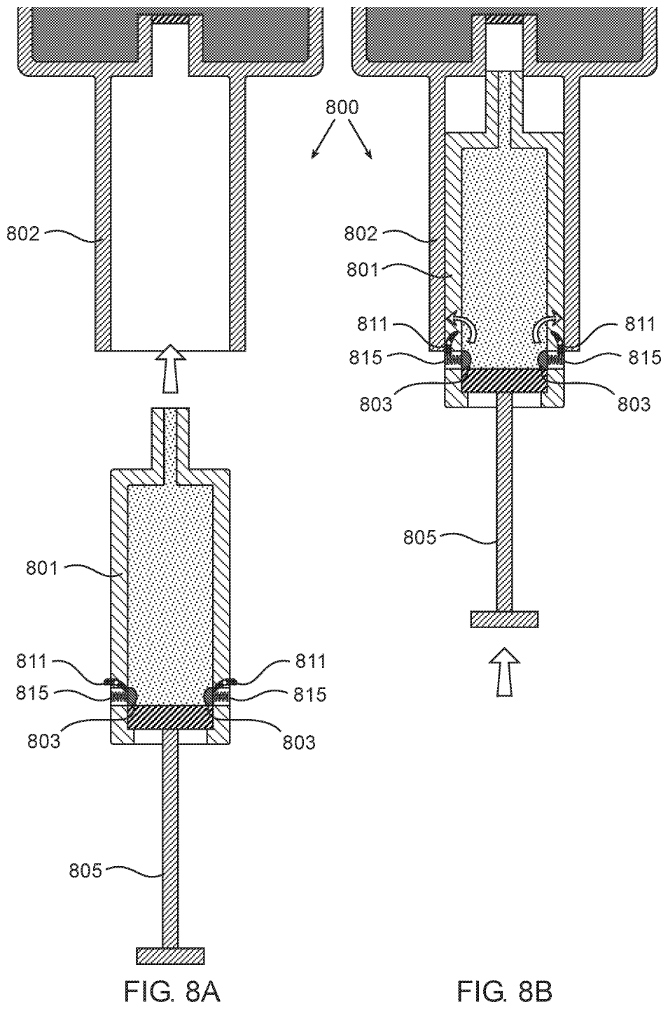

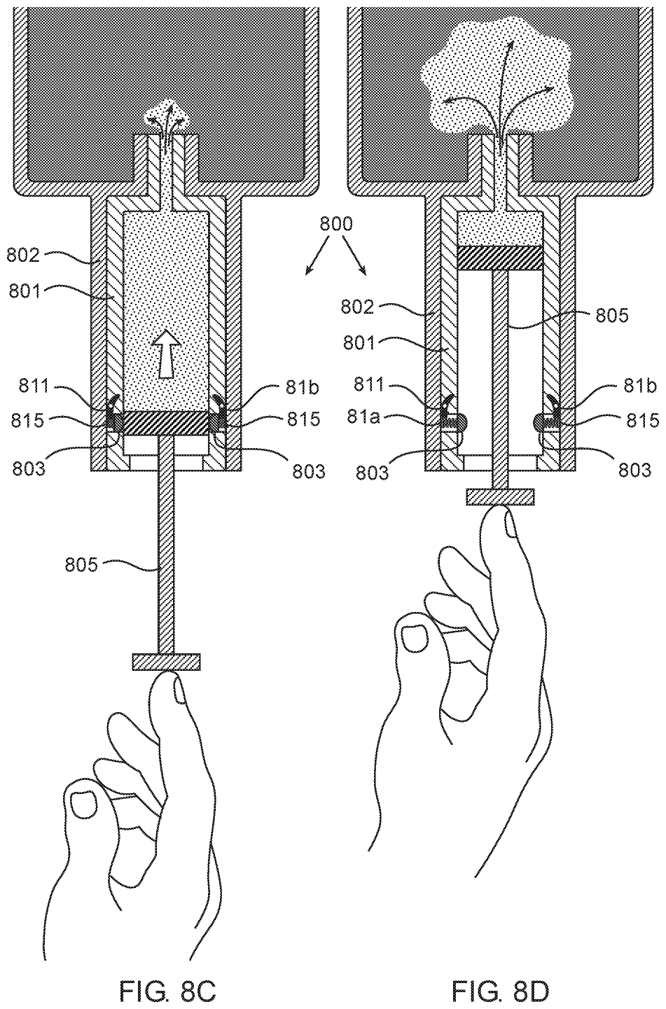

FIGS. 3A-3D illustrate yet another exemplary system 300 for transferring a beneficial/medical substance between a first and a second medical vessels 301 and 302, respectively. System 300 includes locking and unlocking mechanisms, 303 and 311, respectively. Lock mechanism 303 is having a retractable ball-like form which may be spring 315 loaded and disposed in channel 313. Here, the unlocking mechanism 311 is disposed to externally protrude from a side wall of syringe 301. Consequently, the entry of vessel 301 into cavity 308 of vessel 302 forcibly pushes/rotates pin 311, releasing the locking mechanism 303 and allowing retraction thereof when a user pushes plunger 305 towards vessel 302 (FIG. 3C). In FIGS. 8A-8D, yet a similar system 800 is shown which includes a dual lock and unlocking mechanisms. The system 800 includes lock mechanisms 803 in both opposing sides of syringe 801. A further dual unlocking mechanisms 811 is incorporated to prevent a premature fluid transfer.

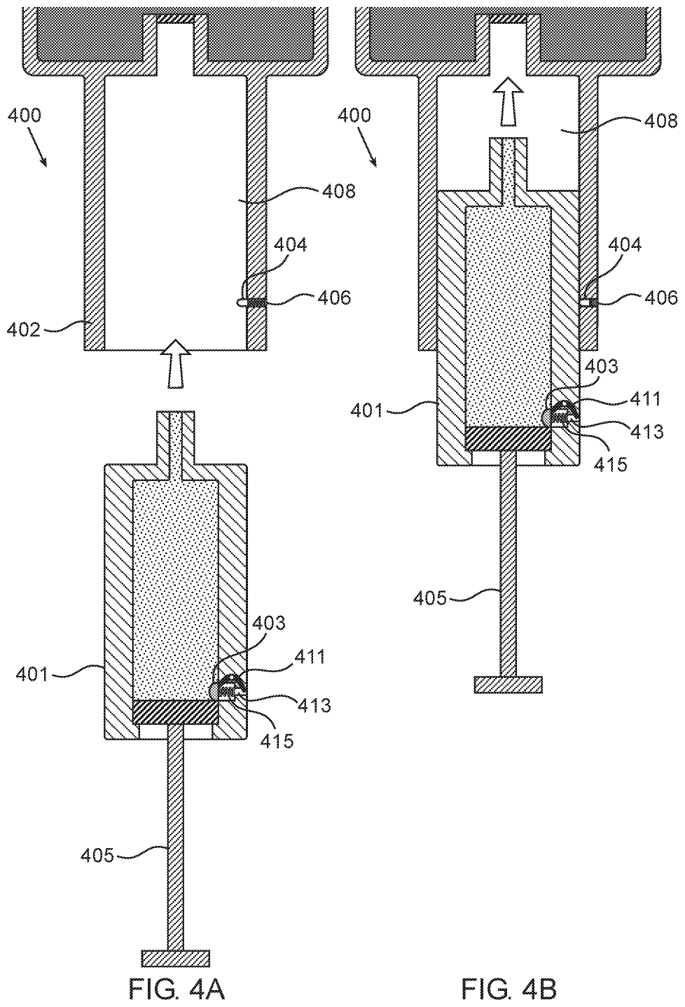

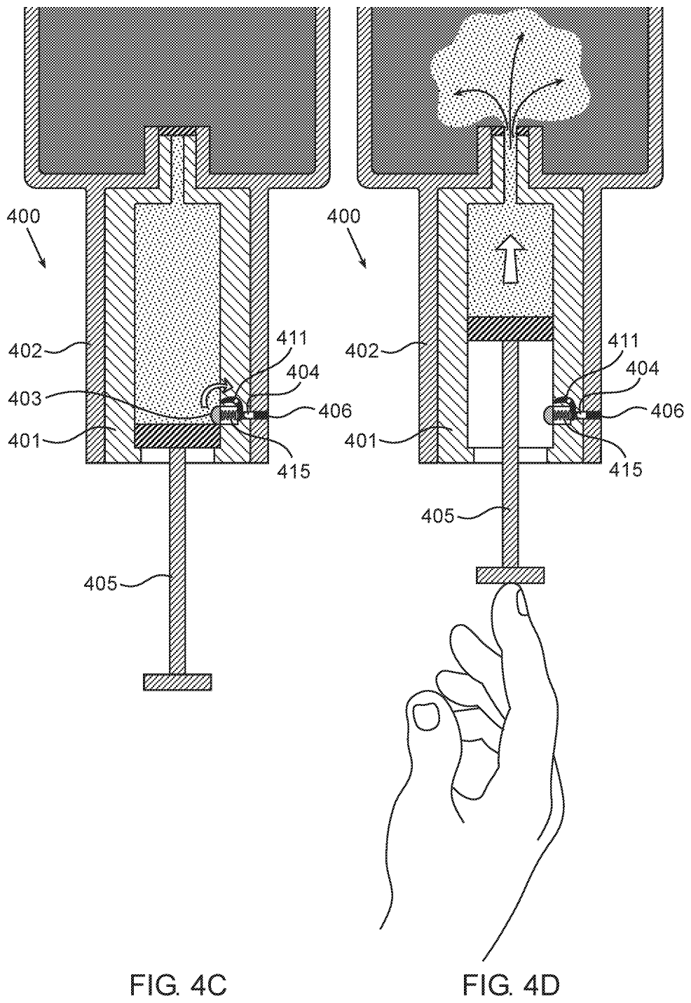

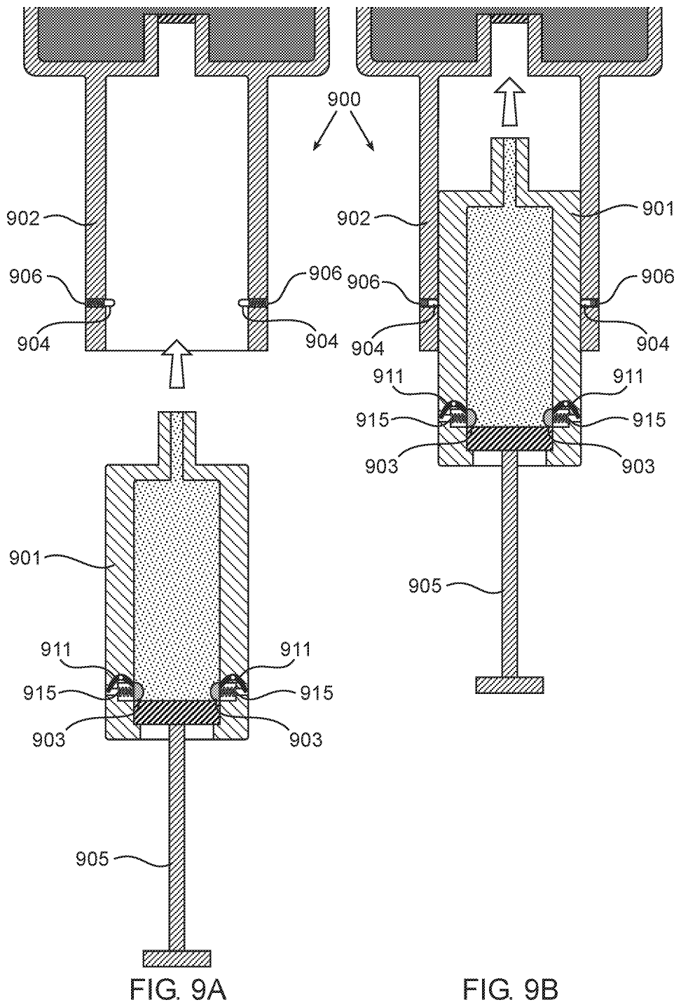

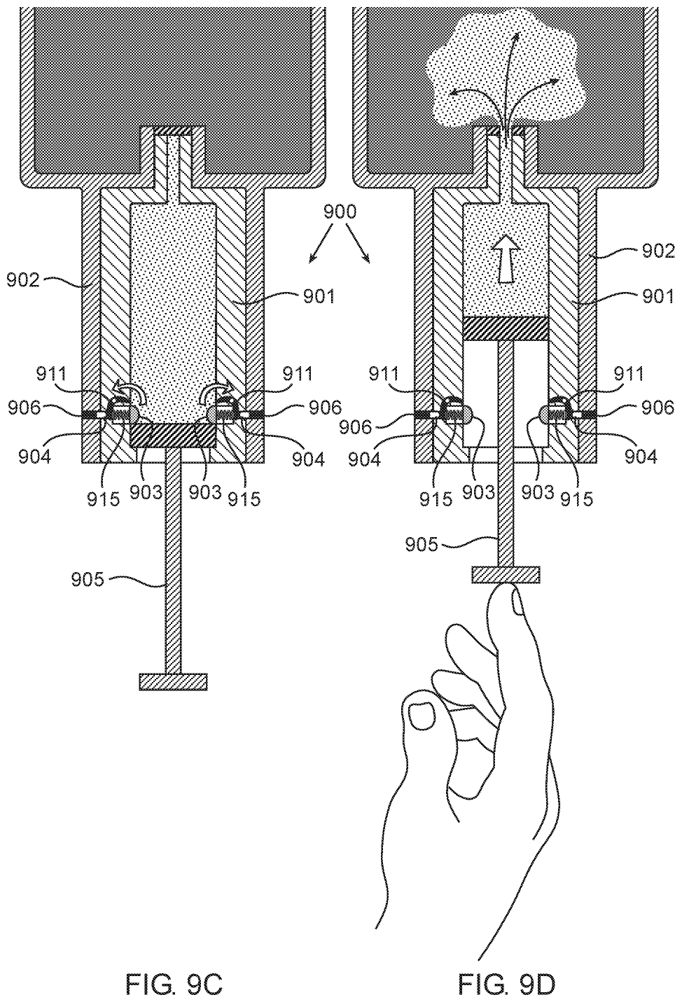

FIGS. 4A-4D illustrate yet another exemplary system for fluid transfer. System 400 includes a lock mechanism in the form of a retractable ball 403 connected to or loaded with spring 415. The unlocking mechanism in this case includes hinged pin 411 and spring 406 loaded retractable pin 404 for ensuring the lock position and mediating the unlocking of the lock mechanism 403. Hinged pin 411 is disposed entirely within a side wall of vessel 401 while retractable pin 404 is connected to a side wall of vessel 402. In the lock position (FIGS. 4A-4B), the unlocking mechanism 411 is connected to, or in contact with retractable ball 403 and prevents retraction thereof such to prevent movement forward of plunger 405. During entry of vessel 401 into cavity 408 of vessel 402, the pin 404 is pushed into the side wall of vessel 402 (FIG. 4B), to allow the insertion of syringe 401. Following a final positioning of the syringe 401 in a cavity 408 of vessel 402 (FIG. 4C), pin 404 can be expanded such to enter channel 413. The entrance of pin 404 into the channel 413 pushes/rotates the unlocking mechanism 411, releasing locking mechanism 403. A user can then push plunger 405 upwardly and inject the beneficial substances/medicament into second vessel 402 (FIG. 4D). In FIGS. 9A-9D, yet a similar system 900 is shown which includes a dual lock and unlocking mechanisms. The system 900 includes lock mechanism 903 in both opposing sides of syringe 901. An unlocking mechanism 911 is further provided to prevent premature fluid transfer. The unlocking mechanism further includes retractable pin 904 loaded with springs 906.

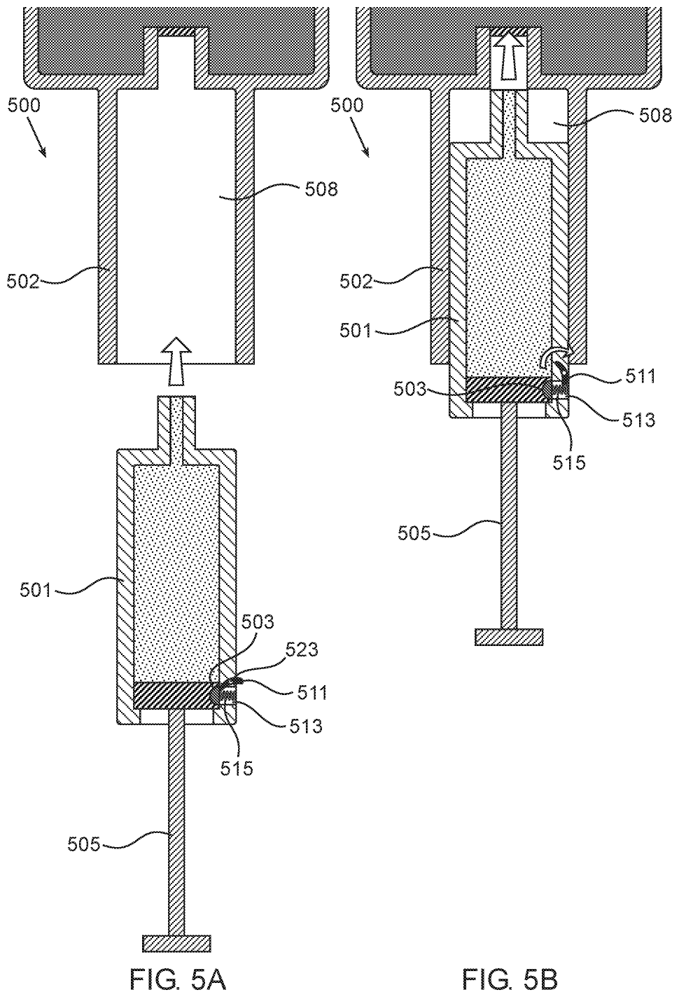

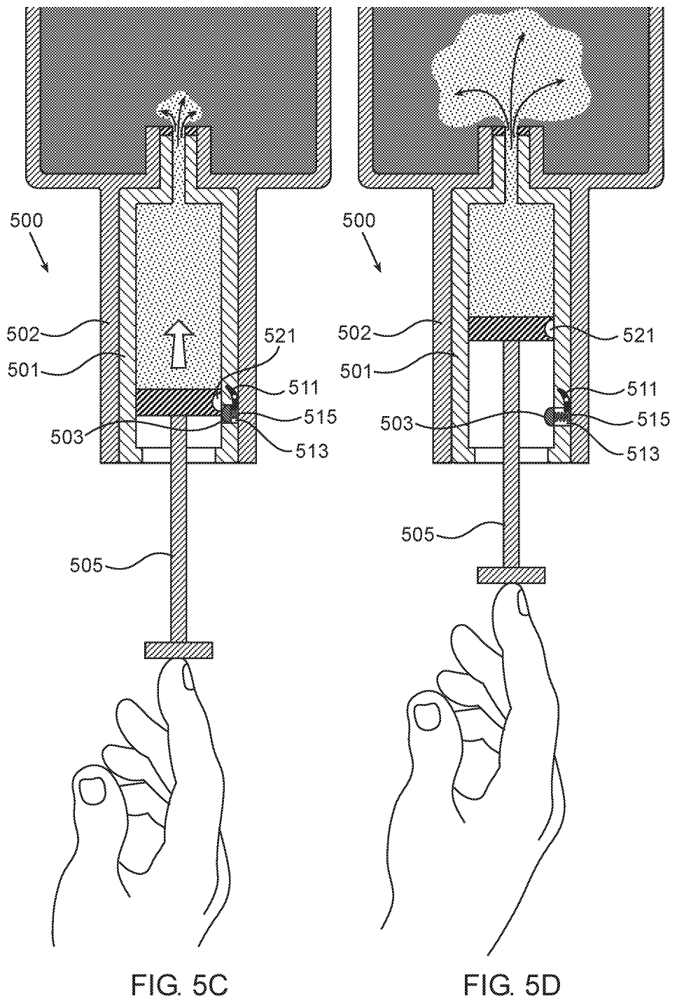

FIGS. 5A-5D illustrate exemplary system 500 wherein plunger 505 includes a dent 521 (FIGS. 5C and 5D) into which lock mechanism 503 extends, thereby locking the plunger 505 when the unlocking mechanism 511 locks lock mechanism 503. The unlocking mechanism includes a hinged pin 511 disposed partially in a side wall of vessel 501 and partially protrudes therefrom to the external environment. Such configuration allows to push the protruded portion of the unlocking mechanism 511 and move/rotate thereof via hinge 523 when inserting vessel 501 into cavity 508, to thereby release lock mechanism 503 (FIG. 5B) and allow injecting the medical substance. Retractable ball 503 is retracted into channel 513 via retraction of spring 515 (FIG. 5C). The plunger 505 can be pushed by a user to release the medical substance (FIG. 5D).

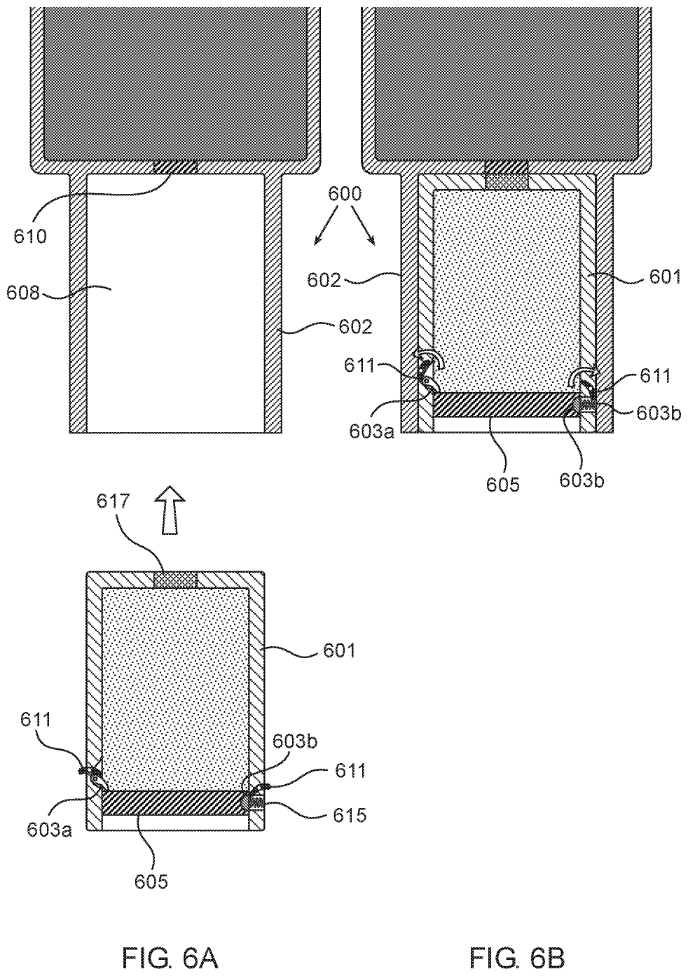

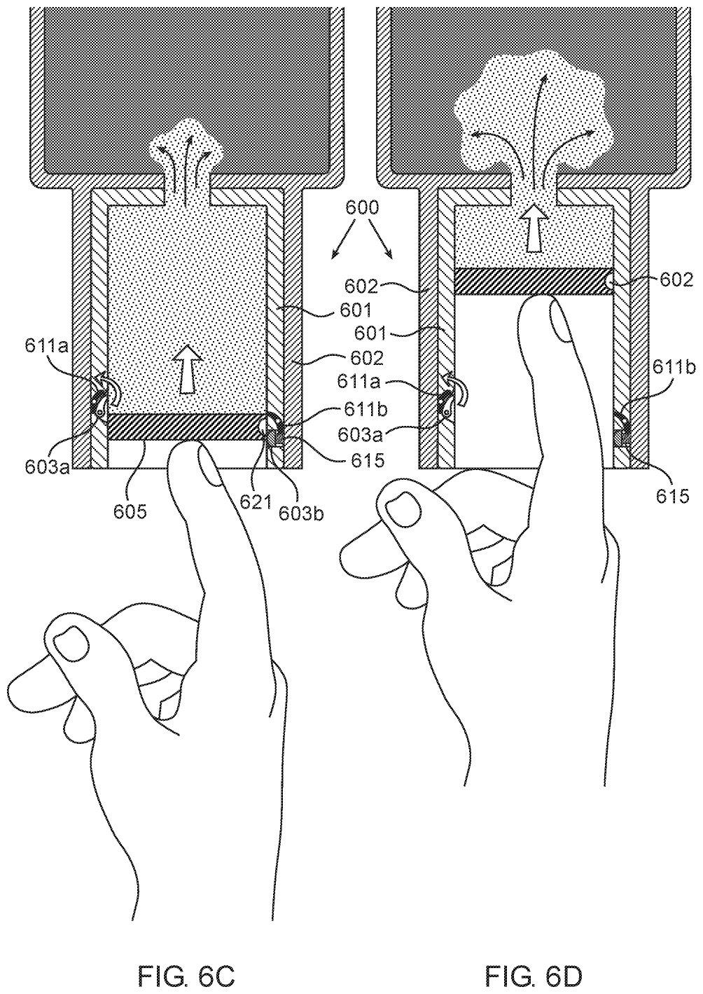

FIGS. 6A-6D show system 600 for fluid transfer between a first vessel, here cartridge 601 and a second vessel 602. Various forms of first and second vessels are contemplated, such as a bag, a vial, an infusion line, etc. In one example, first vessel 601 may be a vial, bottle, or syringe and second vessel 602 may be a bag, bottle, or container. System 600 includes a dual lock mechanism having a combination of two forms of lock mechanisms, being hinged pin 603a and retractable ball 603b. An unlocking mechanism in the form of pins 611 sustains the lock mechanisms 603a and 603b, in the lock positions until the engagement between first and second vessels 601 and 602, respectively, occurs. The engagement/coupling between the vessels forcibly moves the unlock mechanisms 611 (FIG. 6B) and releases the lock mechanisms 603a and 603b, such that a user can now push plunger 605 and allow a fluid transfer between the vessels (FIGS. 6C and 6D).

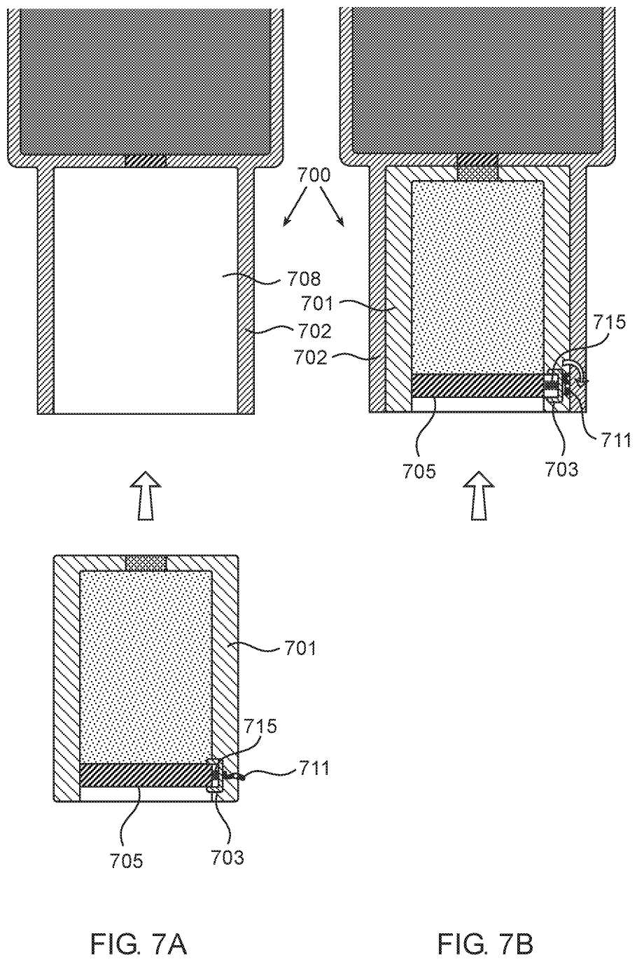

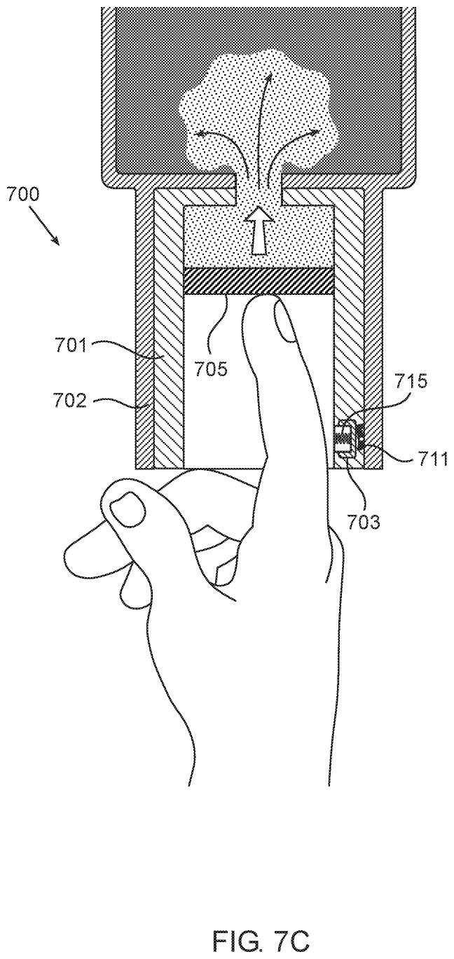

FIGS. 7A-7C illustrate system 700 with a cartridge 701 accommodating a medication. The cartridge 701 has a lock mechanism in the form of a latch 703 such that the plunger 705 is kept in place (locked in place) by the latch locking mechanism 703. Latch 703 is attached to spring 715 which is kept contracted as long as unlocking mechanism 711 is in the lock position (FIG. 7A). The unlocking mechanism in the form of hinged pin 711 is configured to be unlocked by vessel 702 upon the engagement/coupling between the vessels 701 and 702. The unlocking mechanism 711 is shown extending through a wall of the cartridge 701 and exposed to an exterior portion of the cartridge 701. The entry of vessel 701 to cavity 708 involves pushing and/or rotation of the protruding portion of unlocking mechanism 711. In turn, the latch 703 is released by allowing the expansion of spring 715 within the side wall of vessel 701, causing withdrawal of latch 703 into a side wall of cartridge 701 (FIG. 7B). A user can now push plunger 705 forward/upward and allow a fluid transfer between the vessels (FIG. 7C).

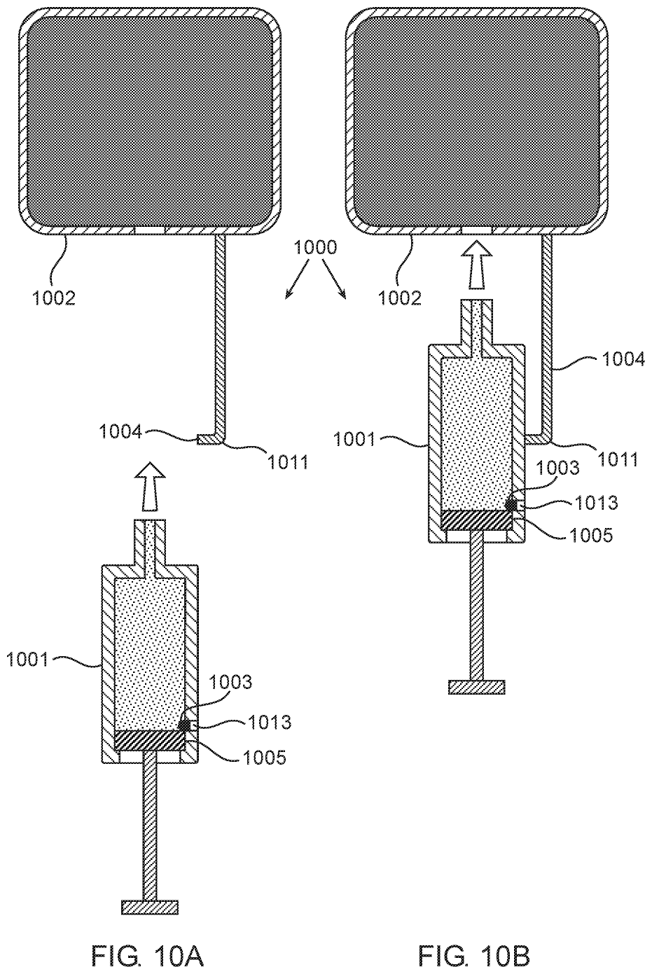

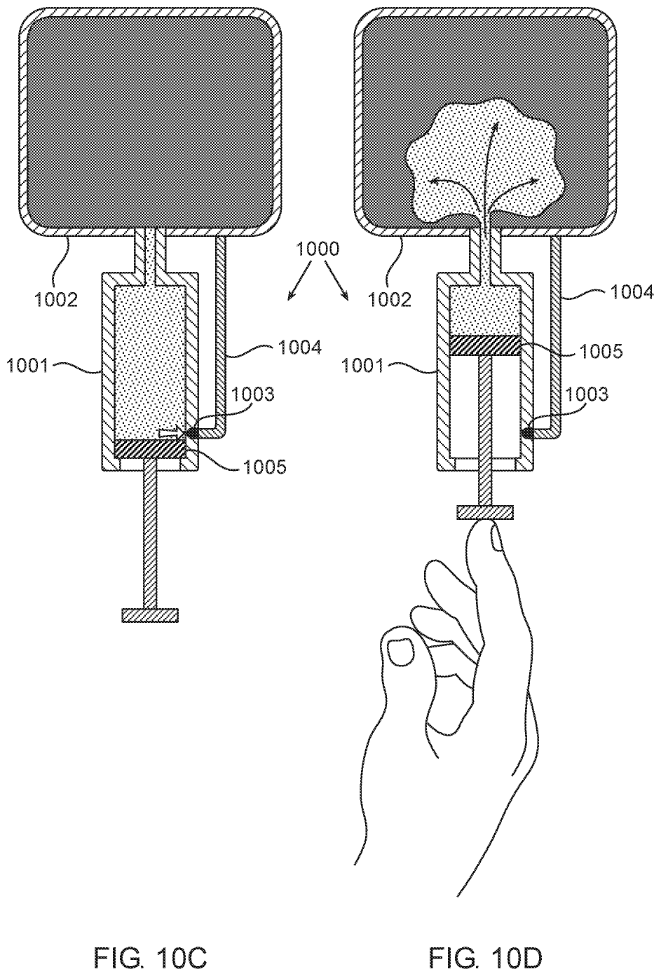

FIGS. 10A-10D illustrate system 1000 with a lock mechanism having a magnetic ball 1003 and an unlocking mechanism in the form of an elongated arm 1011. The elongated arm 1011 extends from a bottom wall of vessel 1002 and may include a magnet 1004 at least at the bottom end thereof. When vessels 1001 and 1002 engage with each other (FIGS. 10B and 10C), elongated arm 1011 may unlock locking mechanism 1003 via magnetic forces. The elongated arm 1011 enters into channel 1013 and attracts ball 1003 thereto via the magnetic forces, such to unlock the lock mechanism 1003 (FIG. 10C) and allow the fluid transfer (FIG. 10D).

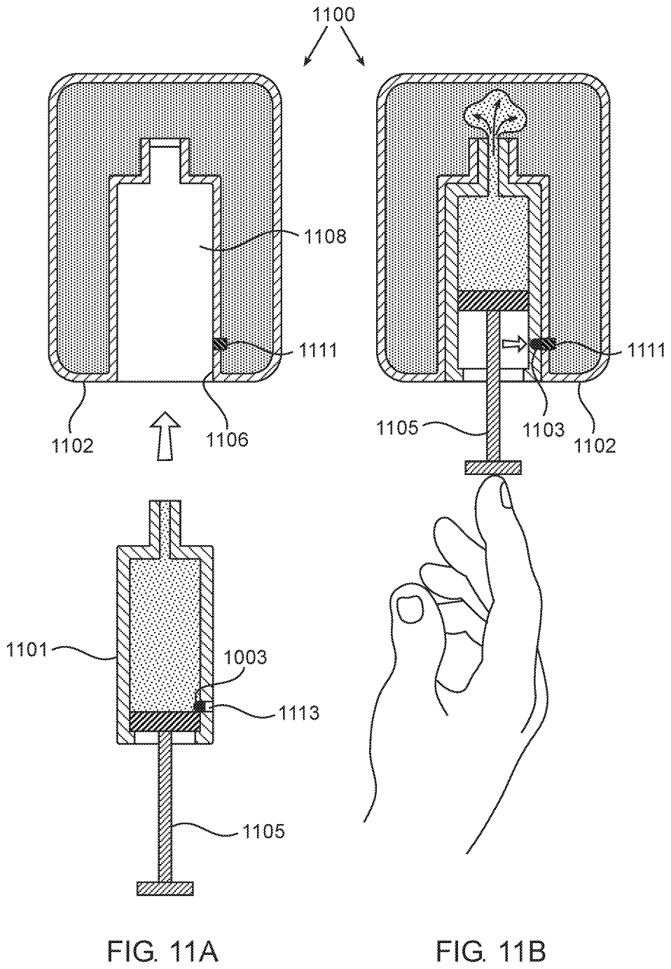

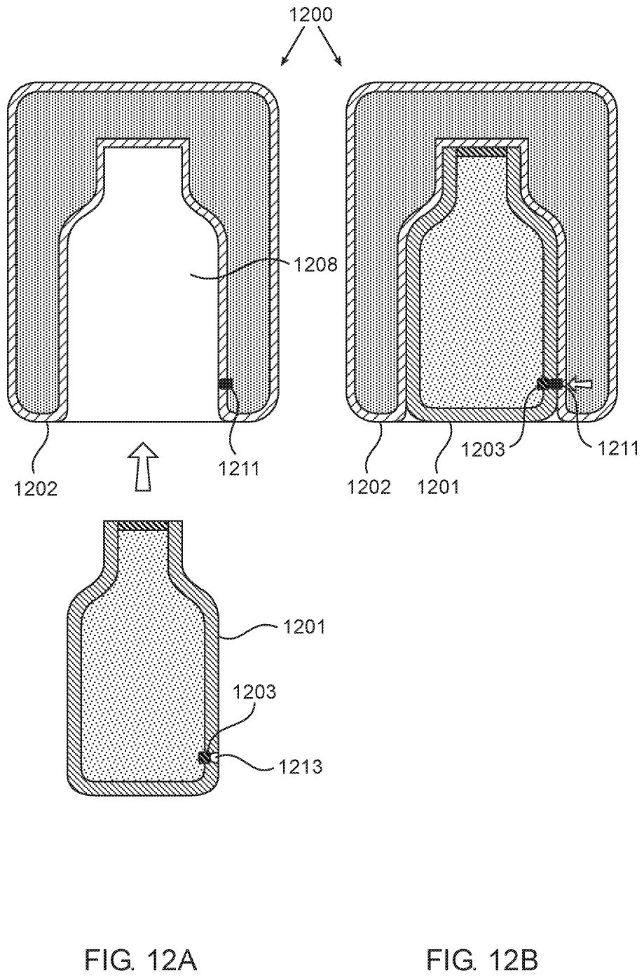

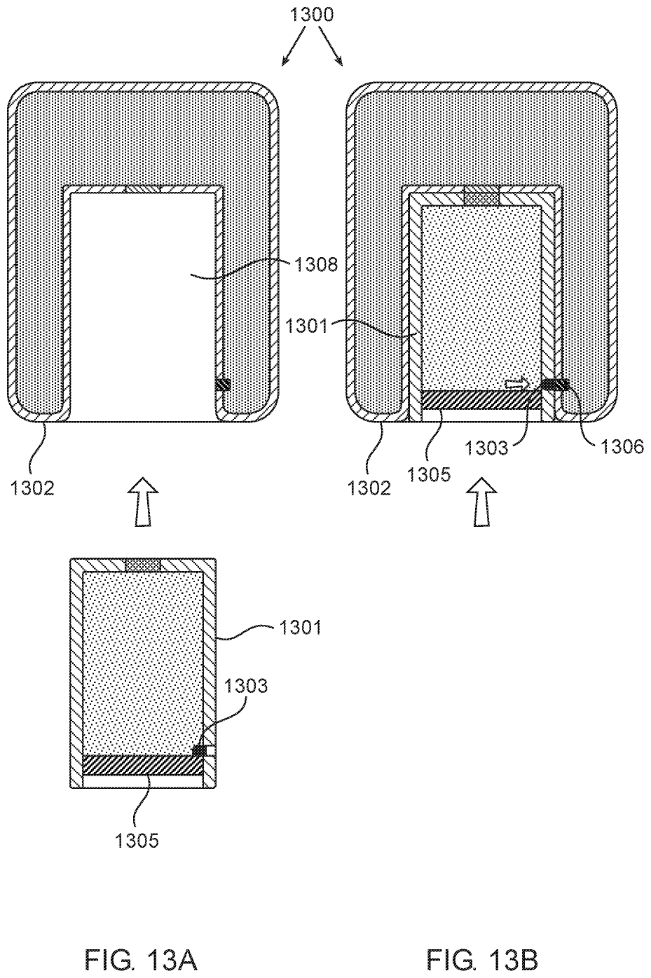

FIGS. 11A-11B illustrate system 1100 which further includes magnets for the lock-unlock mechanisms. Here, unlocking mechanism 1111 includes a magnet disposed at a side wall 1106 of vessel 1102 such that lock mechanism in the form of magnetic ball 1003/1103 may be withdrawn into channel 1113 upon the engagement between the vessels 1101 and 1102 and the horizontal alignment between the lock and unlock mechanisms 1003/1103 and 1111. FIGS. 12A-12B illustrate a system 1200 which is similar to system 1100 but here includes a bottle/vial 1201 and a suitable second vessel 1202 which may be a bag or a bottle. Lock mechanism 1203 moves to channel/hole 1213 when the first vessel 1201 engages second vessel 1202. Please note vessel 1202 has a cavity 1208 into which vessel 1201 enters. Cavity 1208 is configured in size and/or shape and/or volume to accommodate the entry of vessel 1201. Also, vessel 1201 is configured in size and/or shape and/or volume to enter cavity 1208 of vessel 1202. FIGS. 13A-13B illustrate a system 1300 which is similar to systems 1100 and 1200 but here the system includes a cartridge 1301 and a second suitable vessel 1302. The above illustrated systems include various forms of first vessels (e.g., 1101, 1201 and 1301) and corresponding second vessels (e.g., 1102, 1202 and 1302) having cavities (e.g., cavities 1108, 1208, and 1308) configured to accommodate the first vessels.

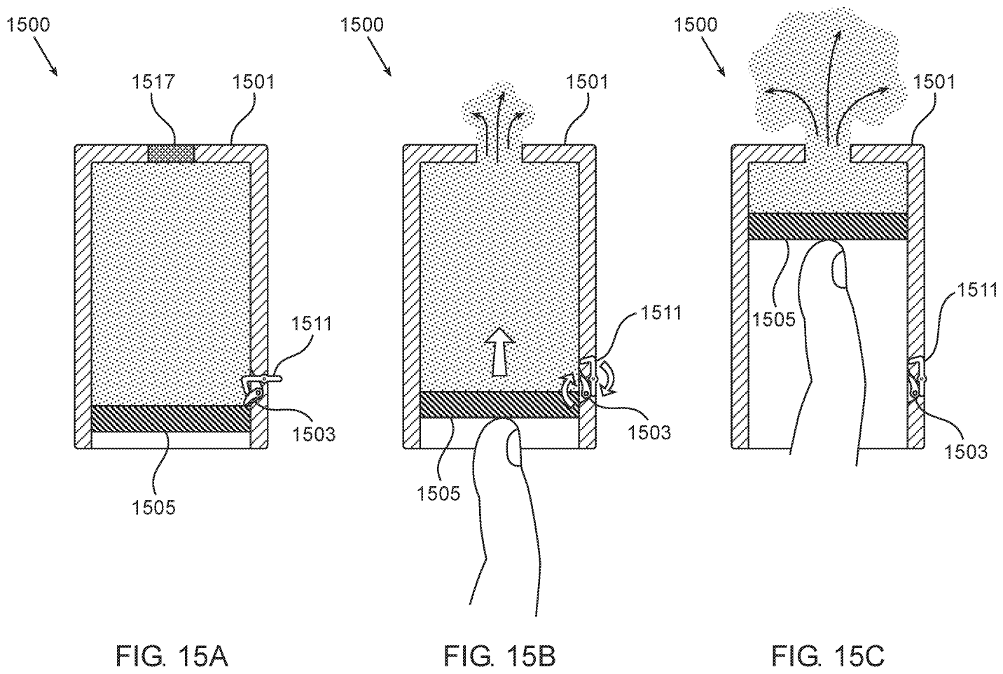

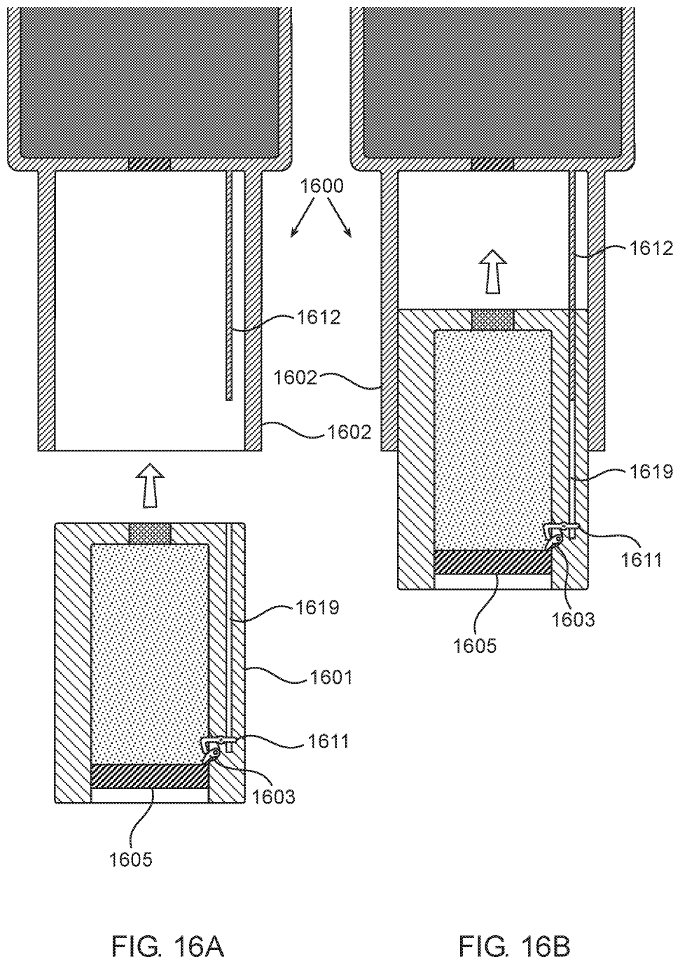

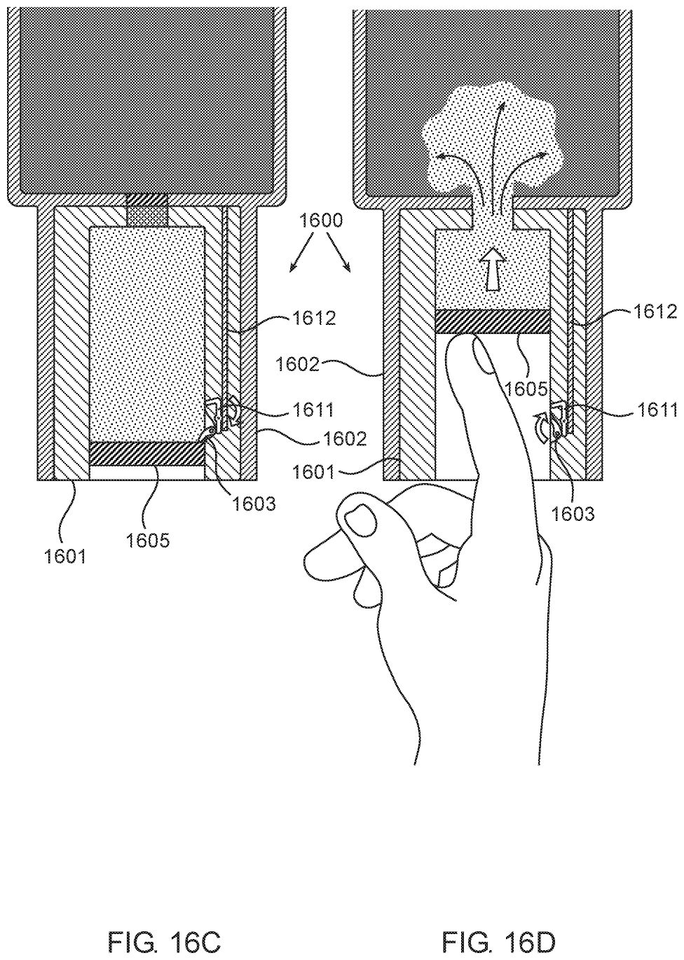

FIGS. 15A-15C illustrate a cartridge 1501 having a lock and unlock mechanisms in the forms of hinged pins 1503 and 1511 which are at least partially protruded from a side wall of the cartridge 1501. Hinged pin 1511 has a hook-like form. In FIGS. 16A-16D a system 1600 is shown which includes a cartridge 1601 with similar pins 1603 and 1611. Here, pin 1611 is disposed/concealed entirely within a side wall of the cartridge 1601. The cartridge 1601 includes a vertical conduit 1619 for accommodating therein an elongated arm 1612. An end of the pin 1611 is disposed to protrude into channel 1619 such that the engagement of cartridge 1601 with vessel 1602 includes insertion of arm 1612 into channel 1619 (FIG. 16B) until reaching pin 1611 and pushing thereof downwardly. The pin is then rotated about 90 degrees vertically and releases pin 1603 (FIG. 16C). A user can then transfer a medicament by pushing the plunger 1605 towards vessel 1602.

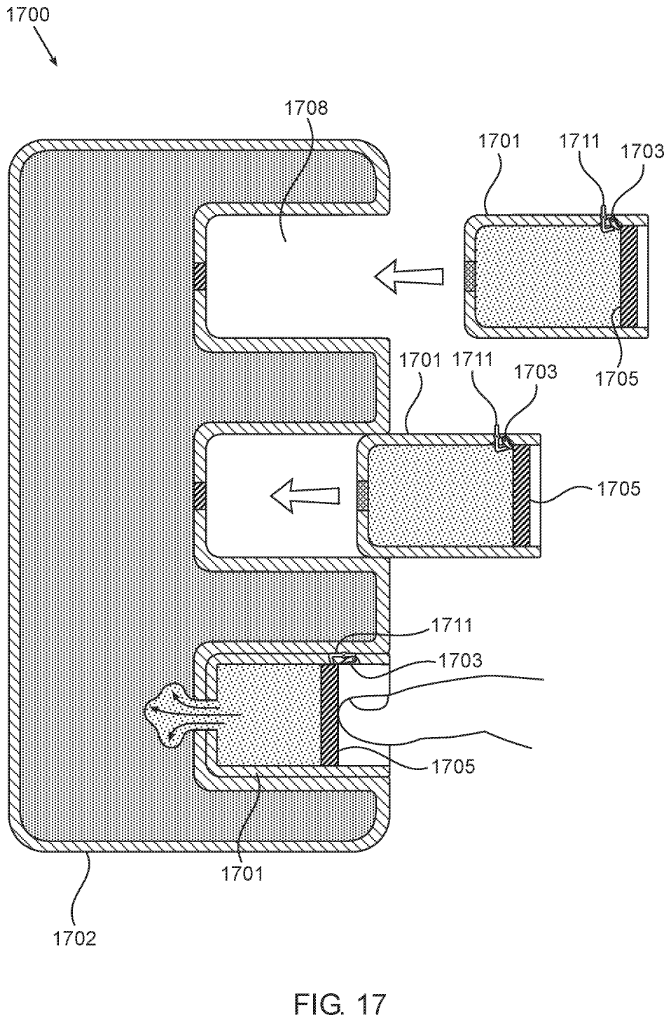

FIG. 17 illustrates system 1700 that includes a plurality of first vessels, being cartridges 1701, and a second vessel 1702, which may be, for example, an infusion bag or bottle that can accommodate the plurality of cartridges 1701. In one embodiment, the vessel 1702 is configured to accommodate one cartridge. In one embodiment, the vessel 1702 is configured to accommodate a plurality of cartridges (e.g., two, three, four or more cartridges 1701). System 1700 includes a lock mechanism with a hinged pin 1703 and an unlock mechanism with hinged pin 1711 that protrudes from a side wall of the cartridge 1701. The unlocking mechanism 1711 unlocks the lock mechanism 1703 when a cartridge 1701 engages vessel 1702 and the side walls of cavity 1708 push the protruding end of the pin 1711. The user can expel fluid form each of cartridges 1701 following the unlocking of lock mechanisms 1703. Optionally, or alternately, in one or more embodiments hinged pin 1711 may directly engage and/or lye adjacent to plunger 1705 (without the presence of pin 1703) thus locking the plunger (not shown). In accordance with this embodiment engagement of vessel 1701 to vessel 1702 rotate/displaces pin 1711 from a locked position to an unlocked position that allows plunger 1705 to move. A similar system 1800 with similar lock mechanisms 1803 and unlock mechanisms 1811 illustrates cartridges in the form of syringes 1801 and cavities having a size and shape that allow the accommodation of the syringes 1801 (e.g., having a syringe-like form).

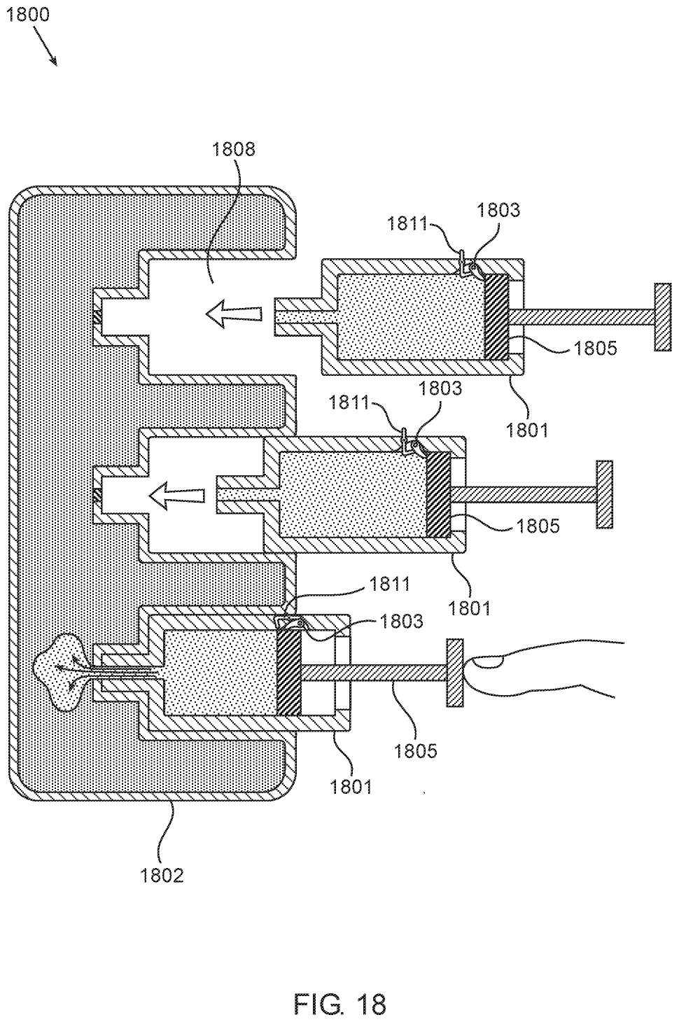

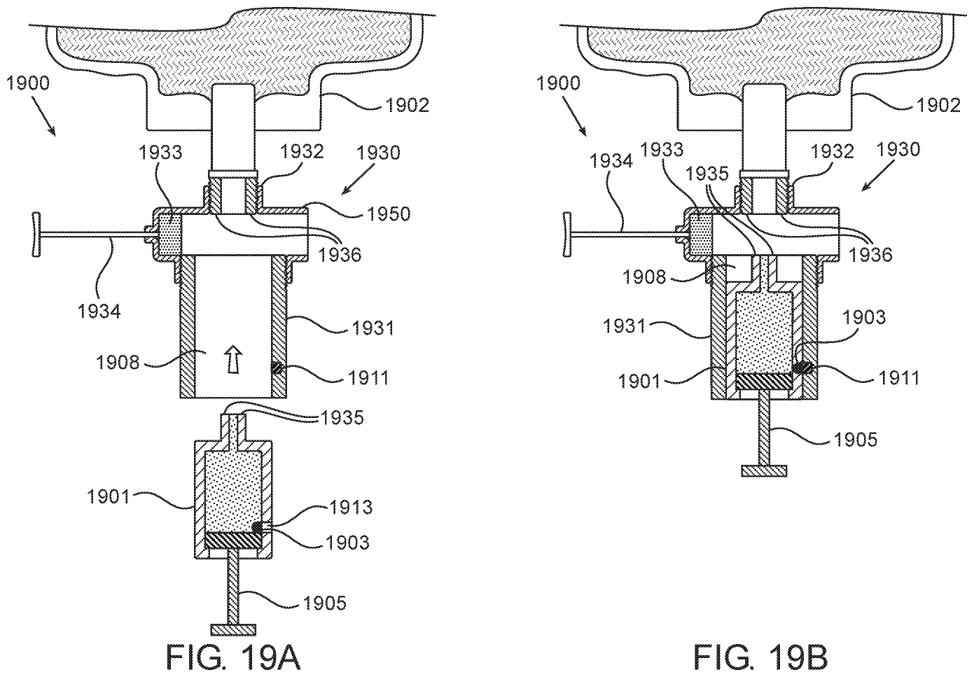

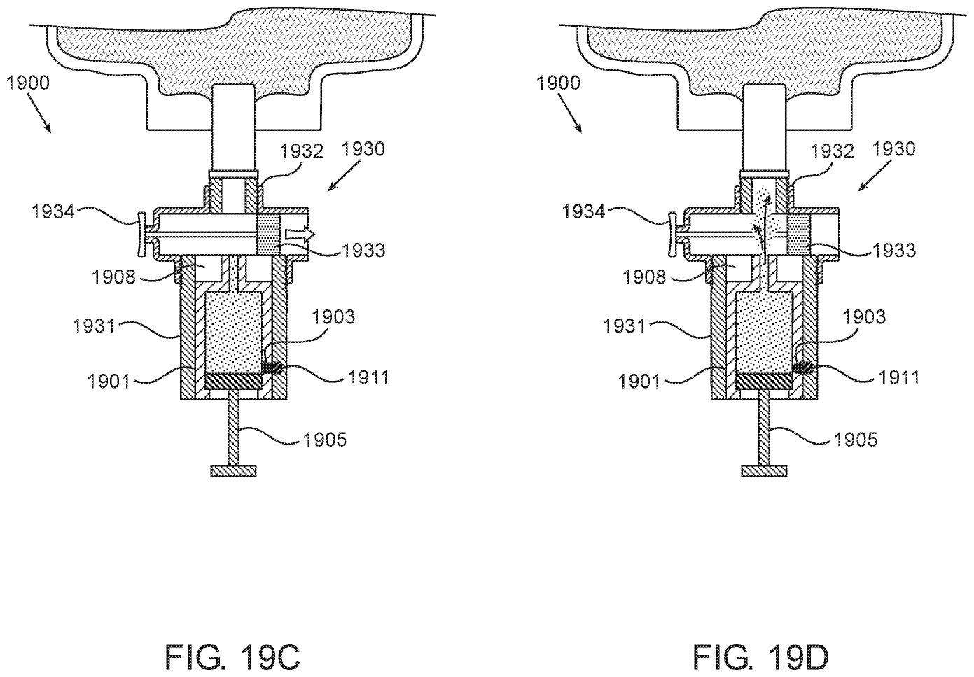

FIGS. 19A-19D illustrate system 1900 for fluid transfer between vessels 1901 and 1902 that includes a decontamination device 1930 having a housing 1950 configured to decontaminate one or more of the surfaces 1935 and/or 1936 of vessels 1901 and 1902. The system 1900 includes a lock mechanism 1903 and an unlocking mechanism 1911 which are similar to those shown in FIGS. 11A-11B. In system 1900, the connection between vessels 1901 and 1902 is via the decontamination device 1930 which incorporates the unlocking mechanism 1911. Decontamination device 1930 includes a first port 1931 which encloses a lumen 1908 for accommodating therein first vessel 1901 or a portion thereof. Decontamination device 1930 further includes a second port 1932 for accommodating therein second vessel 1902 or a portion thereof. The decontamination device 1930 has a moveable wiping member 1933 configured to move across the device 1930 and to thereby decontaminate surfaces 1935 and/or 1936. Wiping member 1933 is manipulated externally to move across the device 1930 via a wiping member actuator having a form of an actuator/handle 1934. Optionally, vessel(s) 1901 and/or 1902 may be provided when already attached, optionally integrally manufactured with device 1930. At about the time of, at the time of, or following the engagement between first vessel 1901 and decontamination device 1930, lock mechanism 1903 is unlocked by the unlock mechanism 1911 (FIG. 19B). Surface 1935 can be decontaminated in parallel, or thereafter the engagement by manipulation of actuator/handle 1934 (FIG. 19C). A fluid transfer may then be performed by a user via plunger 1905. It is important to note that unlocking mechanism 1911 disposed on/in decontamination device 1930 may be (but is not limited to) a pin and/or spring mechanism similar and/or the same as unlocking mechanism(s) described in FIGS. 2A-2D or any of FIGS. 1-18. The locking mechanism of FIGS. 19A-19D may be similar and/or identical to any of the locking mechanisms described in FIGS. 1-18, in which the decontamination device 1930 and/or unlocking mechanism 1911 (of FIGS. 19A-19D) unlocks the locking mechanism(s) of FIG. 19A-19D.

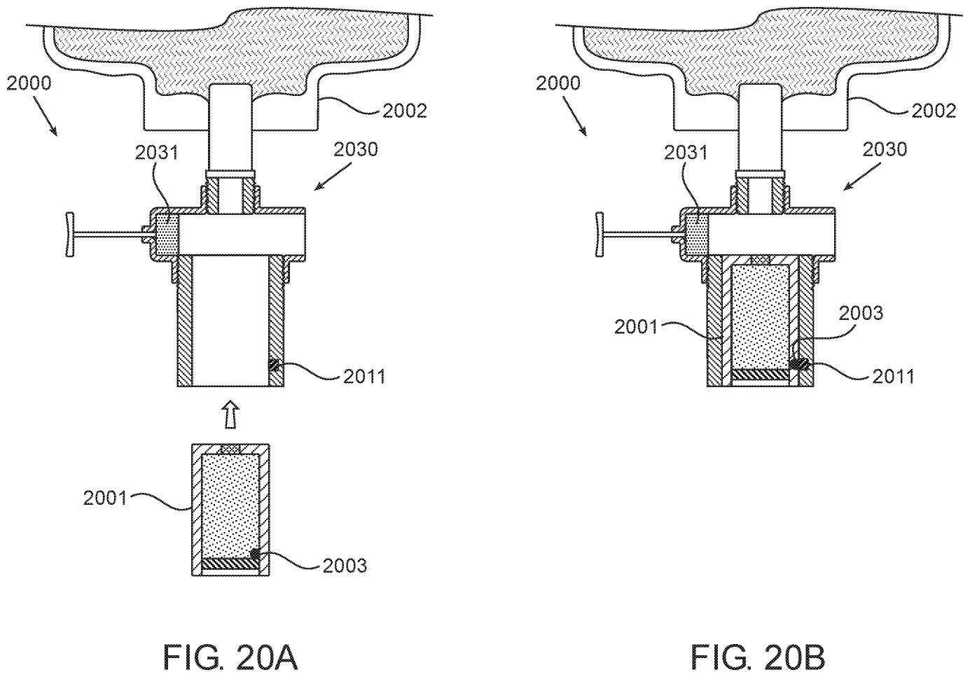

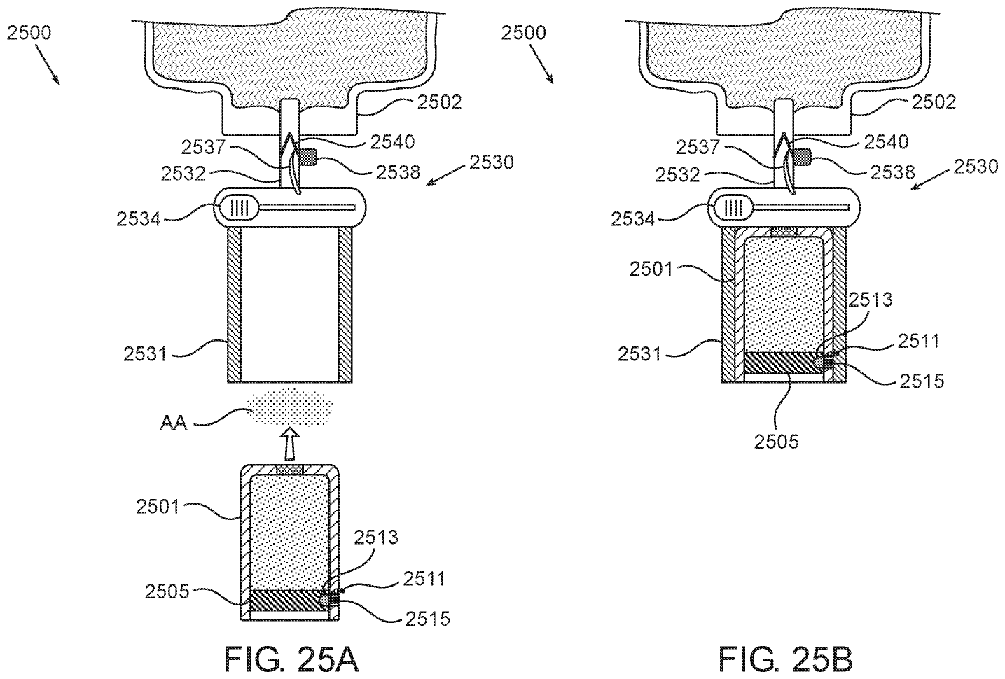

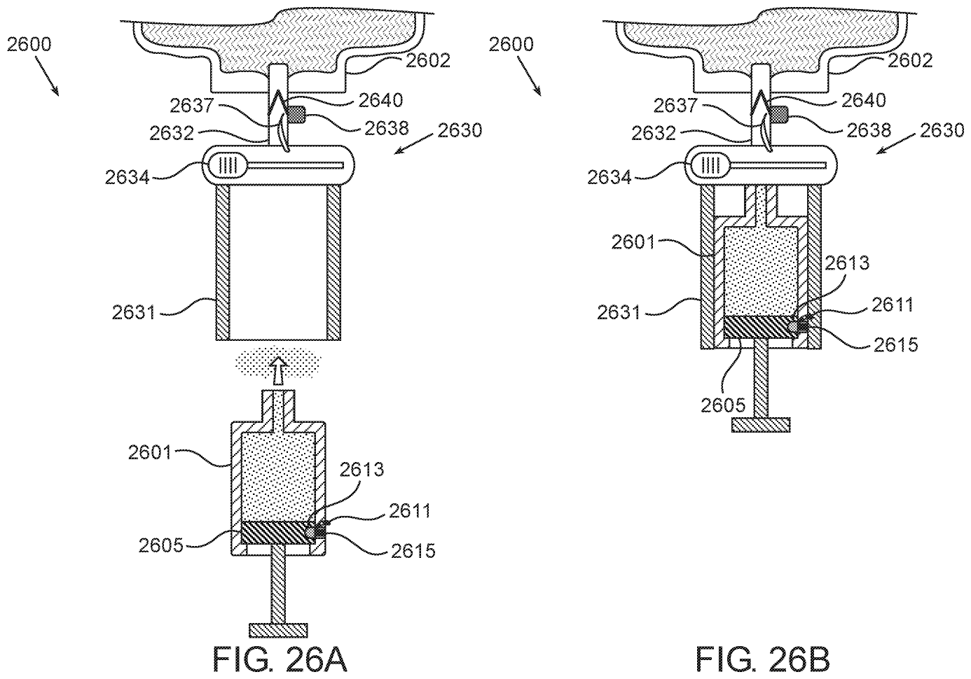

FIGS. 20A-20B illustrate system 2000 which is similar to system 1900 but includes a cartridge 2001 instead of syringe 1901. System 2000 comprises a decontamination device 2030 having a wiper 2031 disposed inside the decontamination device, the wiper 2031 configured to move across the decontamination device to decontamination at least one surface of vessel 2001 and vessel 2002. Unlocking mechanism is disposed on decontamination device 2011, the unlocking mechanism configured to unlock a plunger locking mechanism 2003 of vessel 2001. In system 2500 of FIGS. 25A-25B, the decontamination device 2530 includes a wiping member (not shown) moveable via movement of tab 2534 (tab 2534 is connected to wiping member which is disposed inside decontamination device 2530 through a wall of the decontamination device 2530). A first vessel 2501 in the form of a cartridge includes a plunger 2505 having a dent into which lock mechanism 2513 in the form of a retractable ball extends and can reside in, thereby locking the plunger 2505 when the unlocking mechanism in the form of moveable pin 2511 locks lock mechanism 2513. When first vessel 2501 engages decontamination device 2530, moveable pin 2511 is pressed downwards/displaced/rotates thus releasing/unlocking moveable pin 2511 which allows the retractable ball 2513 to retract into a wall of vessel 2501 which thus allows movement of plunger 2505. A piercing member 2537 may be further incorporated in system 2500, optionally, on port 2532 to pierce a seal or cover (not shown) of second vessel 2502. A valve 2540 may be further incorporated to ensure a one-way fluid passageway. The valve 2540 may be actuated upon a user actuating valve actuator 2538. Piercing member 2537 may be a needle, optionally, a hollowed needle, but various types of piercing members are contemplated, such as members that possess a sharp edge. Steps of establishing a fluid communication between vessel 2501 and vessel 2502 include one or more of: insertion of or connection/engagement between the vessels 2501 and 2502, actuation of the wiping member via tab 2534 thereby effecting a decontamination of one or both vessels, opening valve 2540 by pushing actuator 2538 and transferring a substance by pushing plunger 2505 towards second vessel 2502. The insertion of or connection/engagement between the vessels 2501 and 2502 automatically unlocks the plunger lock mechanism 2513 due to a movement/vertical rotation of pin 2511. The plunger 2505 can thereafter become moveable upon a user pushing the plunger towards second vessel 2502. Yet a similar system 2600 (FIGS. 26A-26B) depicts a first vessel 2601 in the form of a syringe and a second vessel 2602 in the form of an infusion bag. System 2600 is similar to system 2500 but demonstrates a cartridge 2601 in the form of a syringe. The system 2600 includes a decontamination device 2630, a lock mechanism 2613 and an unlock mechanism 2611 in the form of a pin with a spring 2615. A piercing member 2637 is further incorporated to possibly pierce a membrane/covers/seal of second vessel 2602. A valve 2640 and an actuator thereof 2638 in the form of a button/lever may be further incorporated to possibly further monitor the fluid flow between the first vessel 2601 and second vessel 2602.

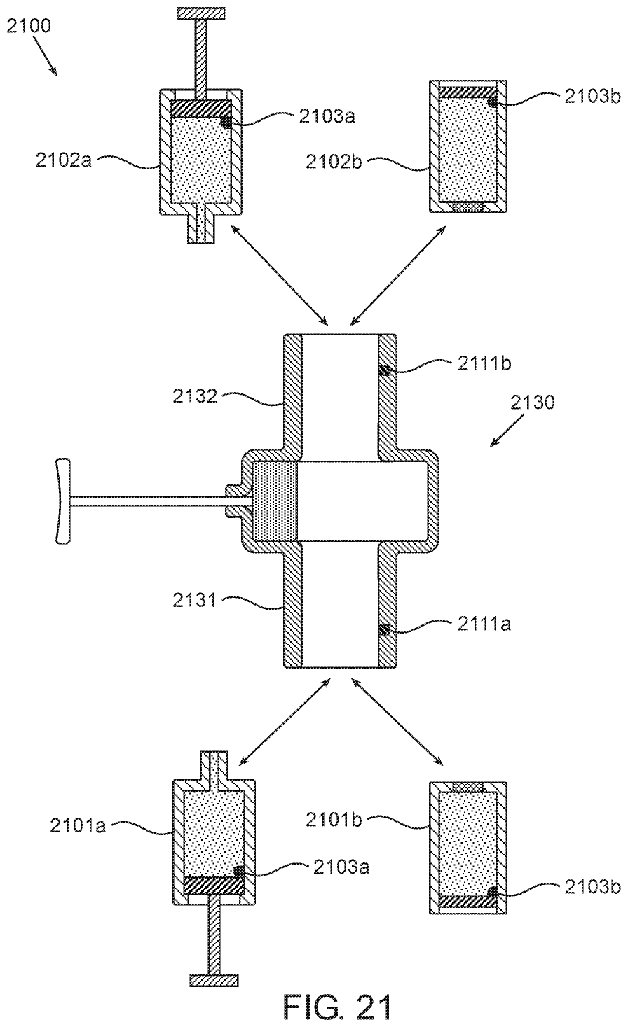

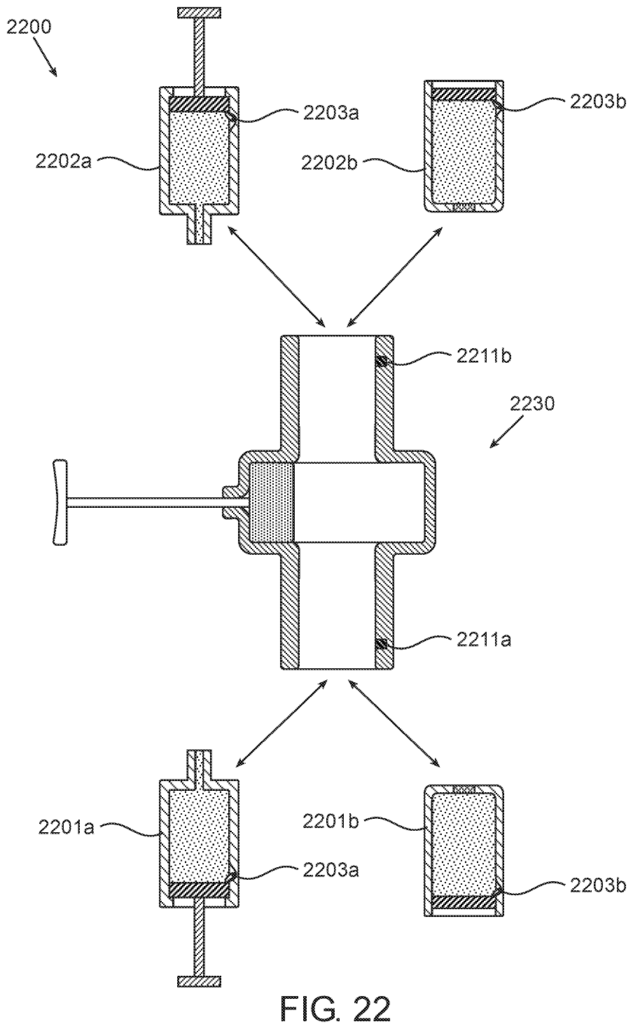

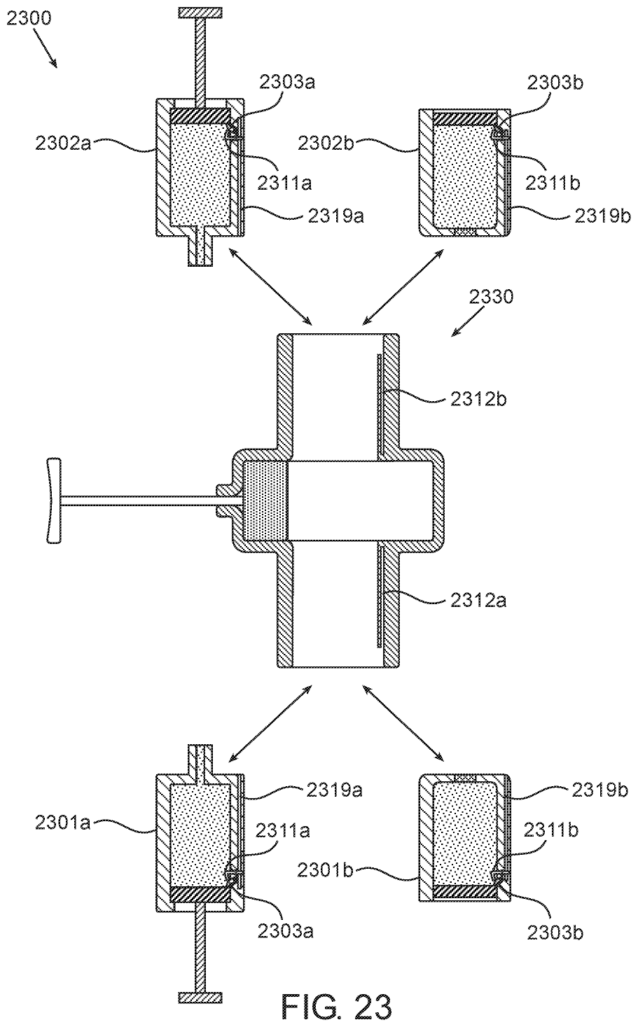

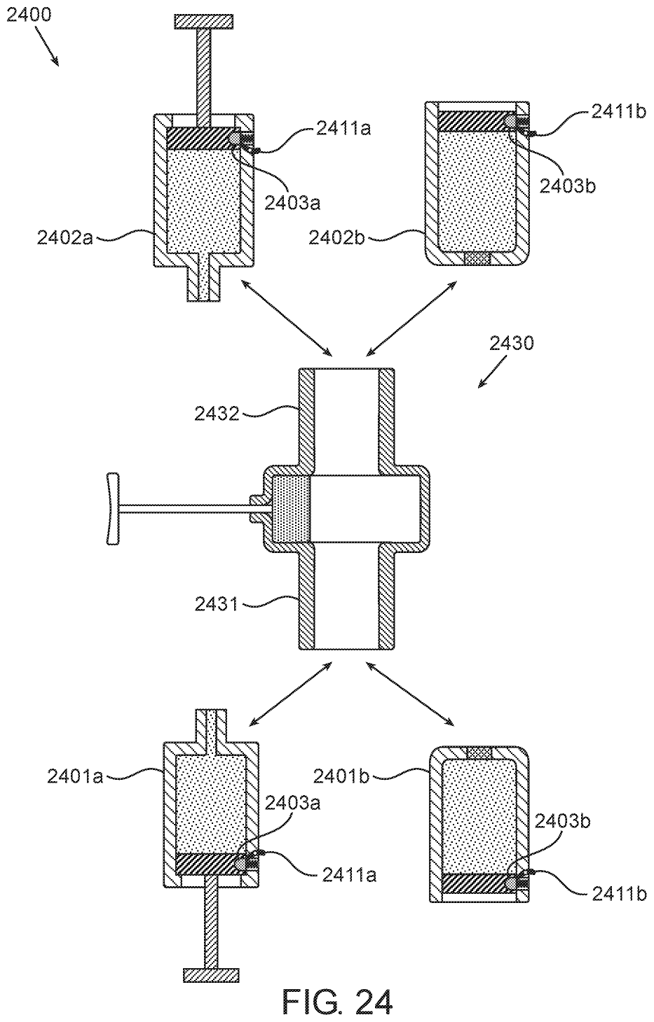

Systems with a decontamination device having a moveable wiping member that can be used as connectors are shown in FIGS. 21-24. The systems 2100-2400 illustrate connectors configured to be coupled to a first and a second vessel and a wiping member that is disposed within the connector and that can wipe off the surface of at least one and/or both vessels. In system 2100 (FIG. 21), the decontamination device 2130 is in the form of a connector and has a first port 2131 and a second port 2132 with an unlocking mechanism 2111a/2111b. The system 2100 further includes a lock mechanism 2103a/2103b disposed on first vessel which may be for example a syringe 2101a or a cartridge 2101b, respectively. A similar lock mechanism 2103a/2103b, or any other lock mechanism as herein described, may be disposed on second vessel; i.e., syringe 2102a or cartridge 2102b. In FIG. 22, a system 2200 with a connector 2230 having a different type of lock mechanism is shown. The lock mechanism is having a form of a hinged pin 2203 which is configured to be unlocked by unlocking mechanism 2211a and/or 2211b of the connector (decontamination device 2230). Please note, the decontamination device 2230 is the same as the connector 2230. The unlocking mechanism(s) 2211a and/or 2211b may be any of the unlocking mechanisms previously described in the specification. Additionally the locking mechanism(s) 2203a and/or 2203b may be any of the locking mechanism(s) previously described in this specification such as, but not limited to, pins, balls, retractable balls, magnets, hinges, latches, and combinations thereof. In FIG. 23, yet another system 2300 having a connector 2330 is shown. The connector is located in the middle of FIG. 23, the connector having a wiping member disposed inside the connector and a handle with a rod disposed through a wall of the connector connecting the wiping member to the handle of the connector. The connector of FIG. 23 is a decontamination device similar to the decontamination device shown and described in FIGS. 19, 20, 21, 22, 24, 25, and 26. FIG. 23 System 2300 includes a lock mechanism in the form of a hinged pin 2303a/2303b and an unlock mechanism in the form of hinged pin 2311a/2311b having a hook-like form and an elongated arm 2312a/2312b which can enter channels 2319a/2319b. In FIG. 24 system 2400, a connector 2430 being decontamination device similar to the decontamination device described in FIGS. 19, 20, 21, 22, 24, 25, and 26, a first vessel 2401a/2401b and a second vessel 2402a/2402b are demonstrated. Here, the lock mechanism is in the form of a retractable ball 2403a/2403b and the unlock mechanism is in the form of a hinged/moveable pin 2411a/2411b. The unlock mechanism(s) 2411a/2411b are configured/adapted to be unlocked at the time, at about the time, and/or after engagement/coupling of vessels 2401a/2401b/2402a/2402b to decontamination device 2430. The decontamination device 2430 unlocks the lock mechanism(s) 2403a/2403b of the vessels.

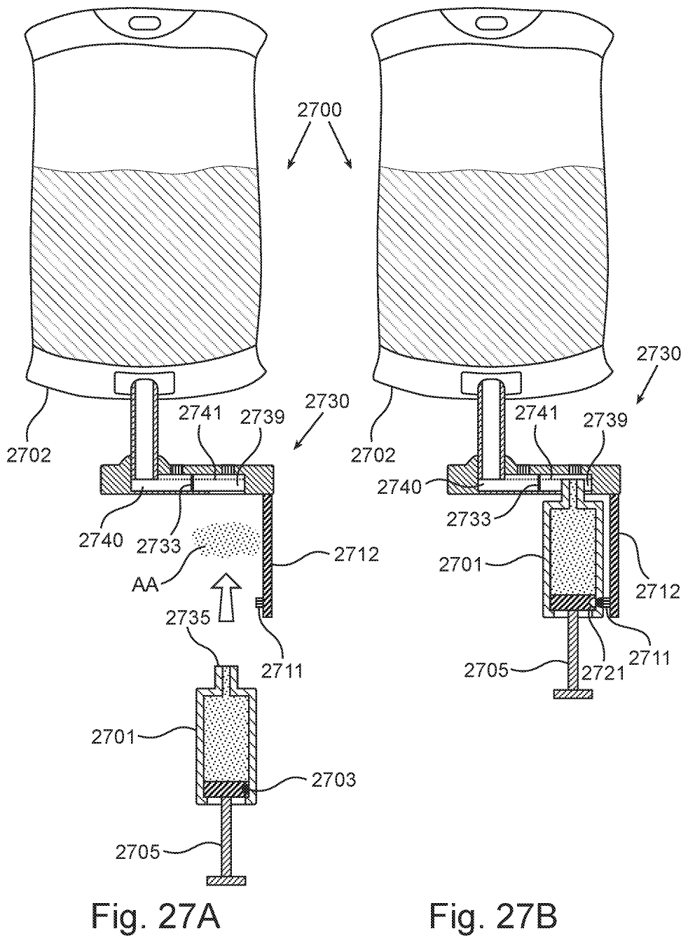

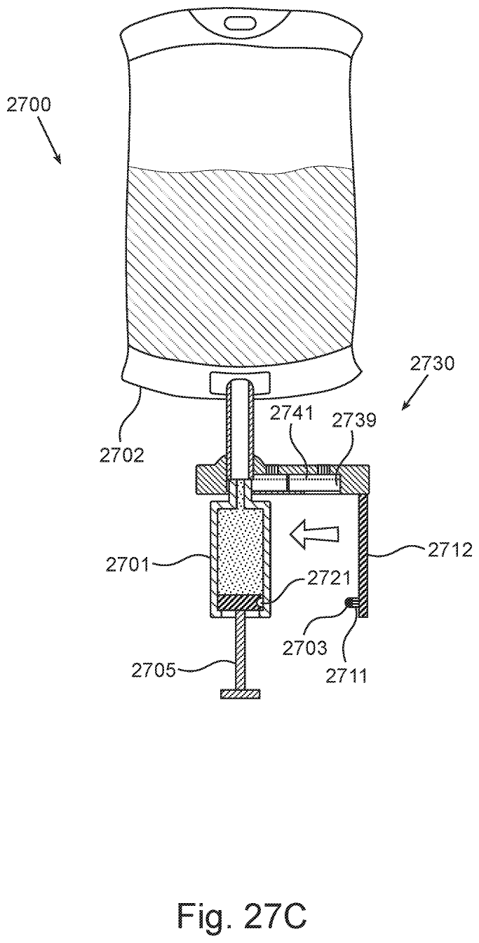

FIGS. 27A-27B illustrate yet another exemplary system 2700 with a decontamination device 2730 having a static wiping member 2733 disposed between a first compartment 2739 and a second compartment 2740 of the device 2730. At least a portion of syringe 2701 is moveable within the device 2730 between a first position in first compartment 2739 (FIG. 27B) and a second position in second compartment 2740 (FIG. 27C). Ambient Air (AA) particles are present between the devices and the wiping member 2733 is configured to decontaminate the surface/seal 2735 upon movement of the vessel 2701 towards second compartment 2740. The system 2700 further includes a lock mechanism in the form of a magnetically-retractable ball 2703 and an unlock mechanism 2711 disposed at an end of elongated arm 2712. The engagement between vessels 2701 and 2702 includes an attachment/contact or proximity between unlock mechanism 2711 and lock mechanism 2703 such that retractable ball 2703 is attracted to a magnet in unlock mechanism 2711 and shifts into a socket (not shown) in a side wall of syringe 2701. The ball 2703 which was initially within a dent 2721 in plunger 2705 is now retraced to the syringe's side wall and the plunger 2705 can be moved forward to expel a medicament. The movement of vessel 2701 within the device may be effected via a rail mechanism 2741 disposed across/in/on device 2730. Unlock mechanism 2711 and lock mechanism 2703 are not limited to the unlock/lock mechanism(s) described in FIGS. 27A-27C. Unlock mechanism 2711 and lock mechanism 2703 may take the form of any of the lock/unlock mechanism(s) described in any of FIGS. 1-26 and/or anywhere in this specification. The unlock and/or lock mechanism(s) of FIGS. 27A-27C may include (but are not limited) to pins, springs, balls, retractable balls, magnets, latches, retractable latches, and combinations thereof.

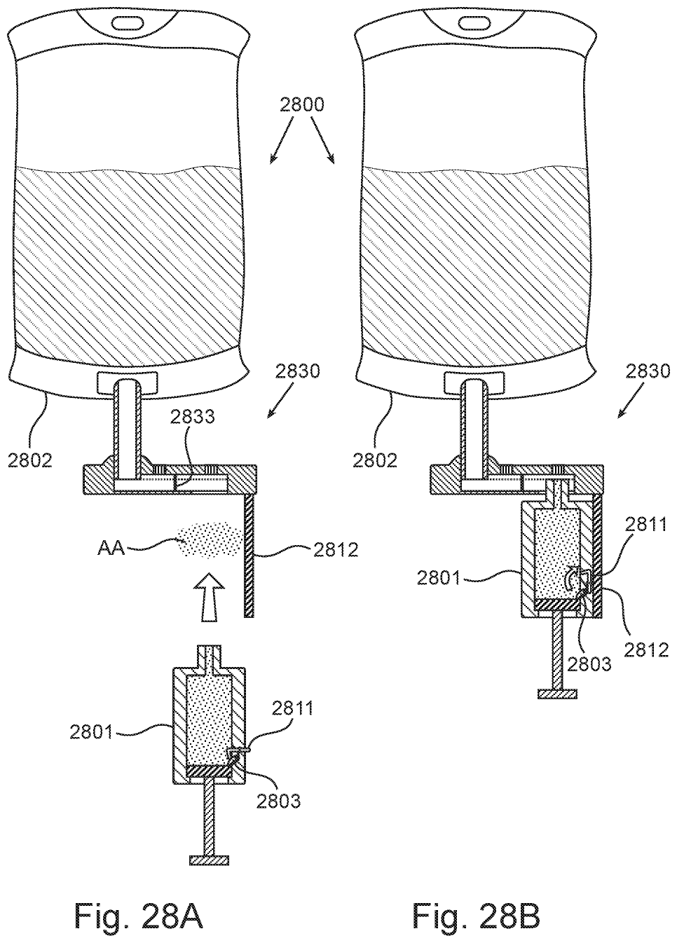

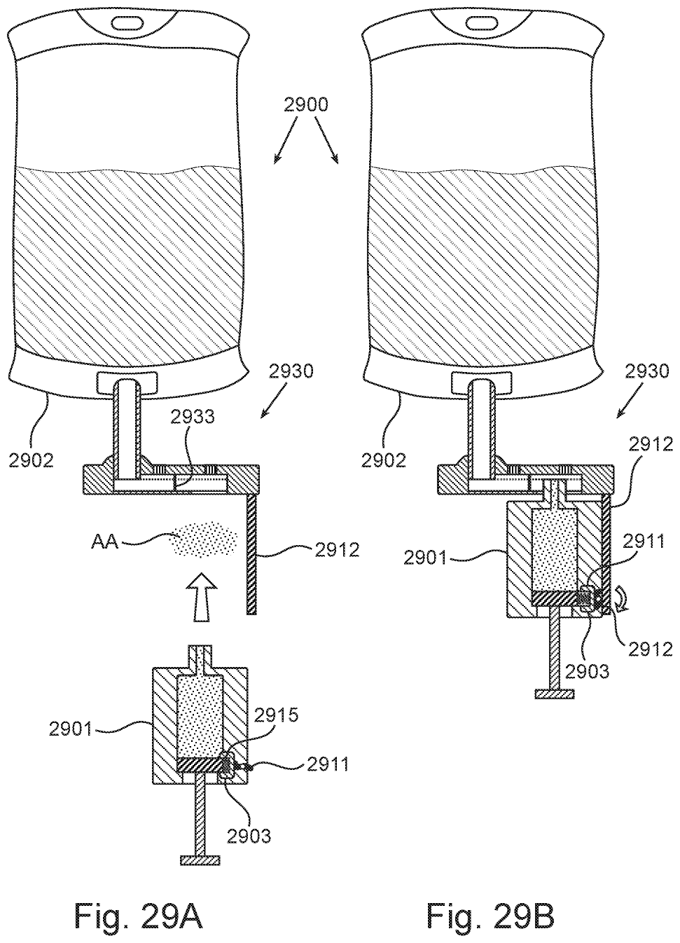

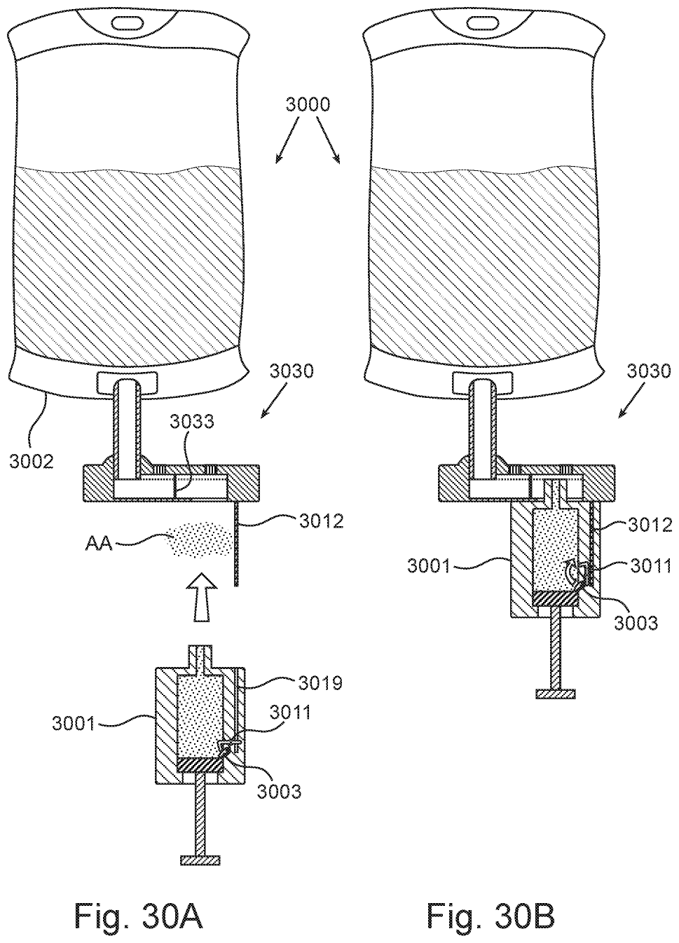

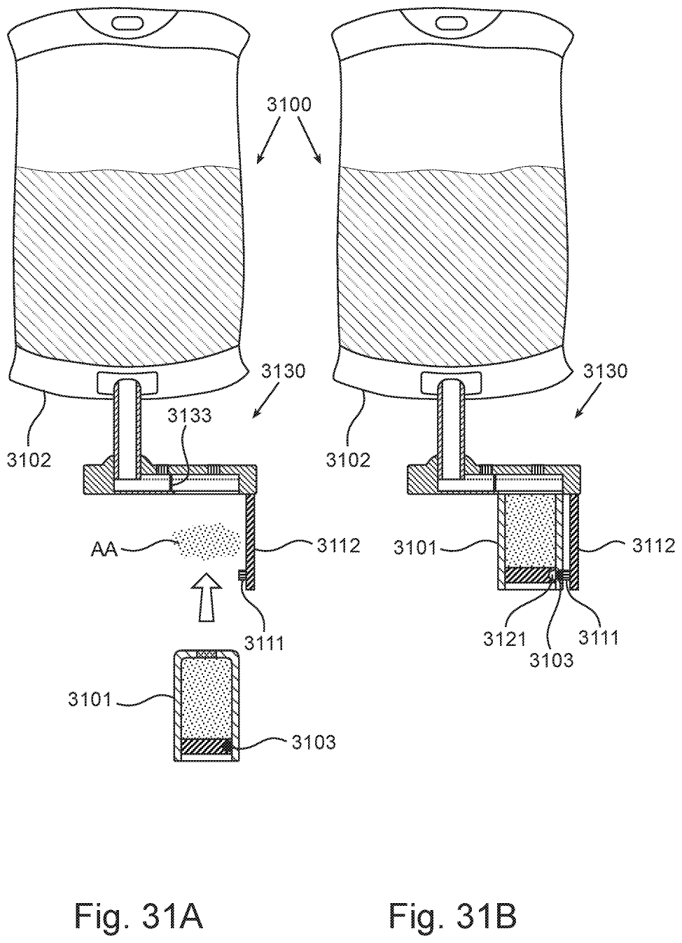

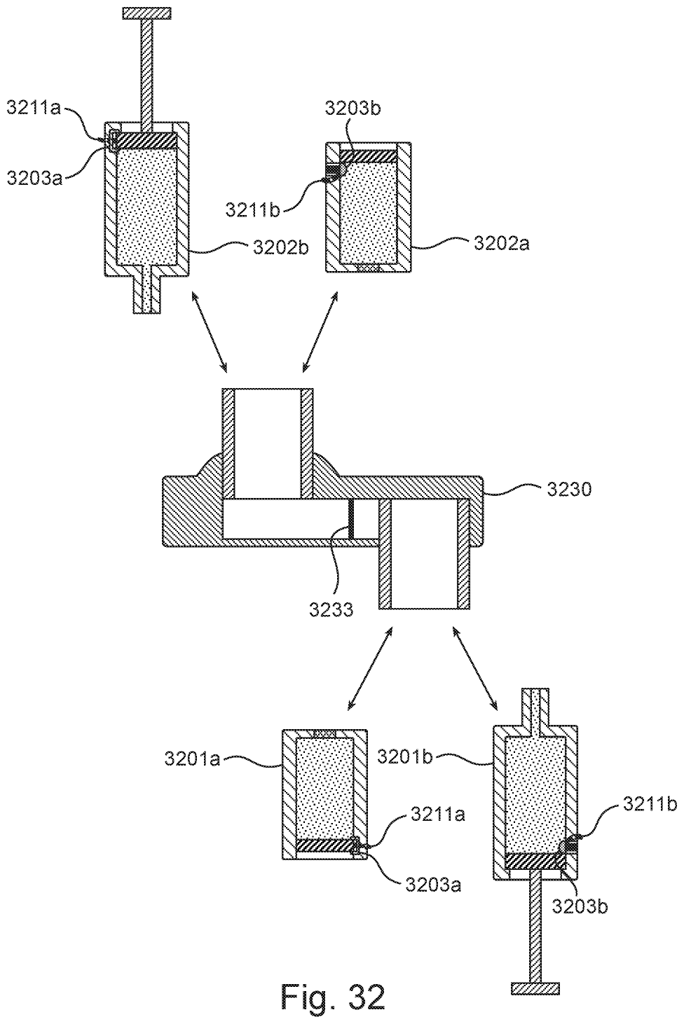

A system 3100 is shown in FIGS. 31A-31B which is similar to system 2700 with the exception that the first vessel is a cartridge 3101. A system 2800 is shown in FIGS. 28A-28B which is similar to the system of FIGS. 27A-27C with the exception that the lock mechanism 2803 is in the form of a hinged pin and the unlock mechanism includes a hinged pin 2811 and an elongated arm 2812. A system 2900 is shown in FIGS. 29A-29B which is similar to systems 2700 and 2800 with the exception that the lock mechanism is in the form of a latch 2903 and spring 2915 and the unlock mechanism includes a hinged pin 2911 and an elongated arm 2912. A system 3000 is shown in FIGS. 30A-30B which is similar to systems 2700, 2800, and 2900 with the exception that the unlock mechanism includes a hinged pin 3011 disposed entirely within a side wall of syringe 3001 and an elongated arm 3012 extends from a bottom wall of decontamination device 3030. Elongated arm 3012 is configured to enter channel 3019 and when doing so to forcibly rotate hinged pin 3011, thereby releasing/unlocking lock mechanism 3003. Channel 3019 is configured in size and/or shape to accommodate entry of elongated arm 3012. FIG. 32 illustrates system 3200 with a decontamination device, in the form of a connector 3230 that can be engaged with various types of first and second vessels. The device 3230 has a static wiping member 3233 that can decontaminate a surface of the first and/or second vessel upon movement thereof within the device 3230. As illustrated, first vessel may be a cartridge 3201a or a syringe 3201b. Similarly, second vessel may be a cartridge 3202a or a syringe 3202b. The first and/or second vessels are moveable within the connector, thereby effecting a decontamination of surfaces of the vessels. Various types of lock-mechanisms are further contemplated for the first and second vessels and may be selected, for example, between a latch 3203a and a retractable ball 3203b, or any other lock/unlock mechanism as herein described. Similarly, various types of unlock mechanisms are contemplated and may be for example a hinged pin 3211a/3211b. Engagement/coupling of the vessels to the connector/decontamination device 3230 unlocks the plunger lock mechanism(s) of the vessels.

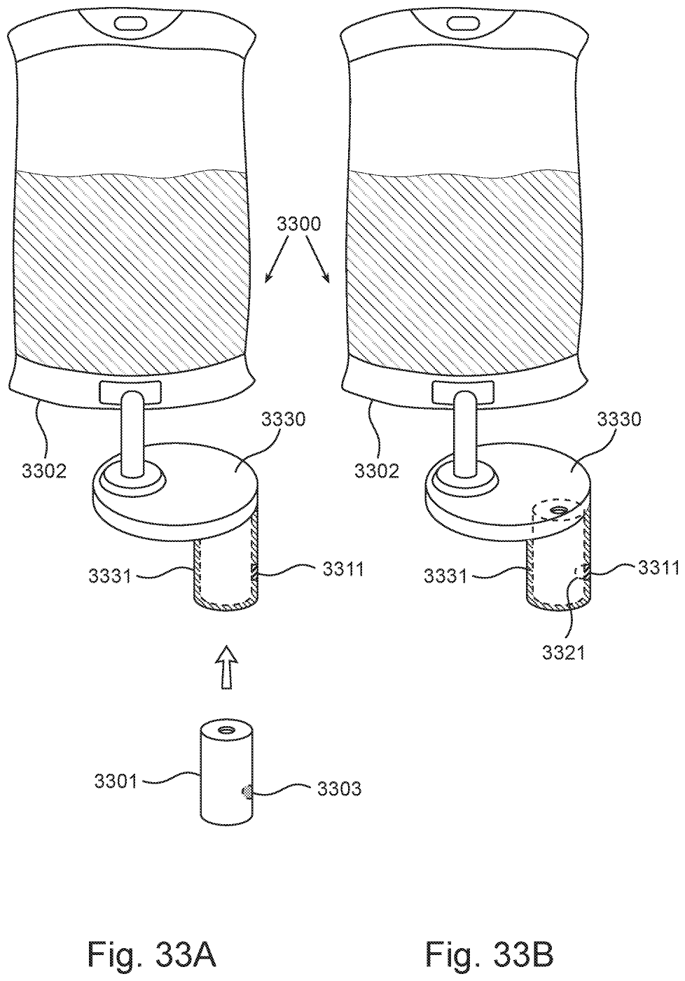

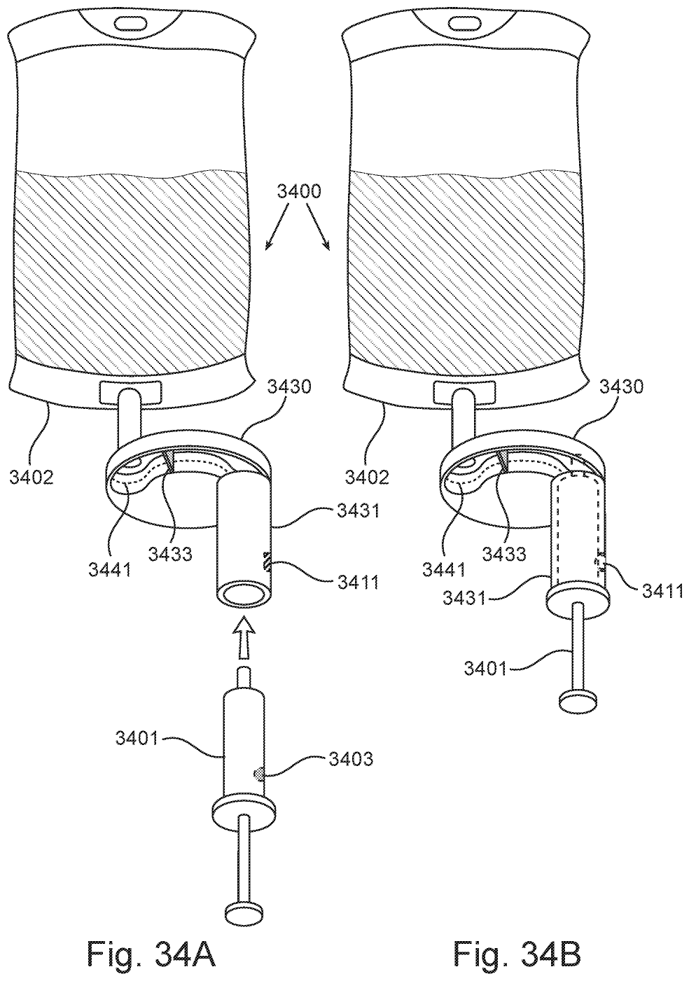

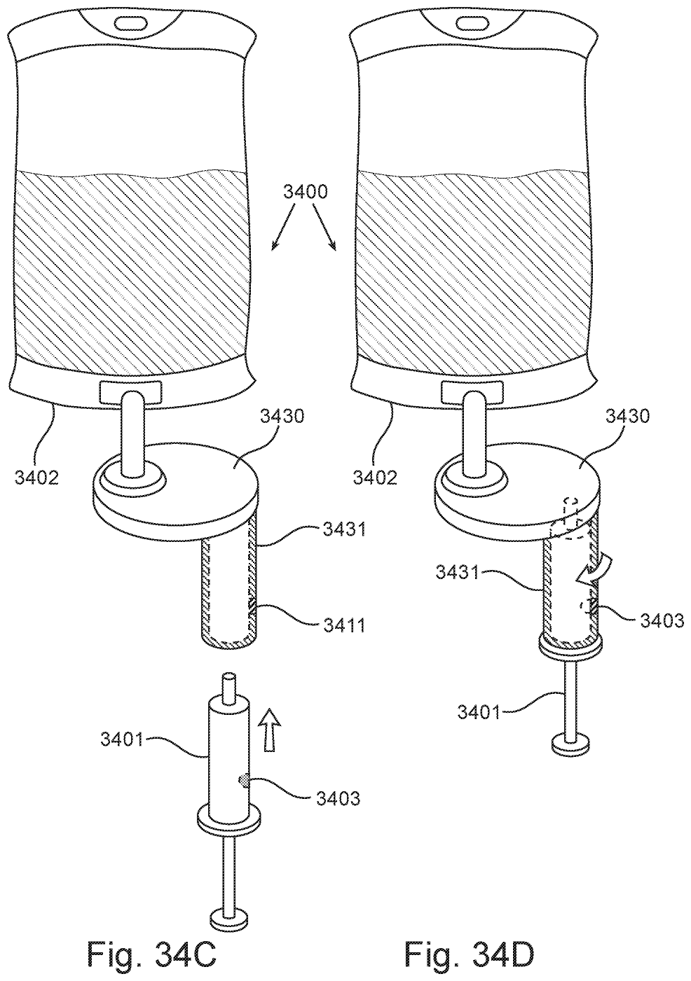

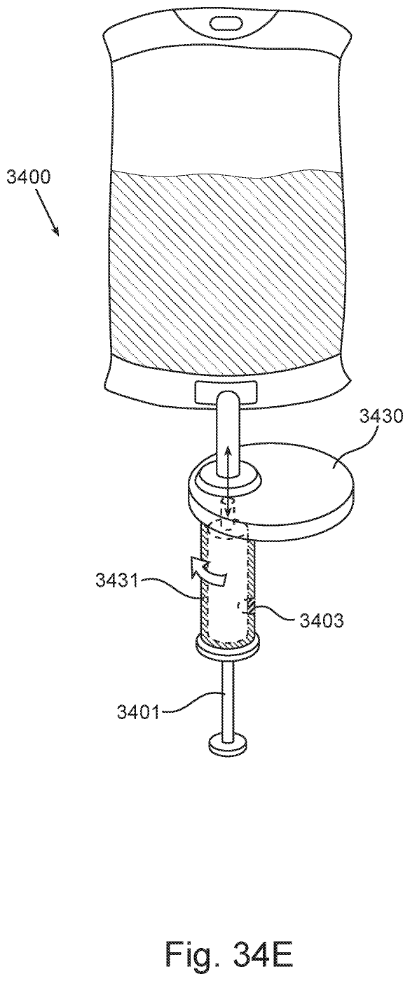

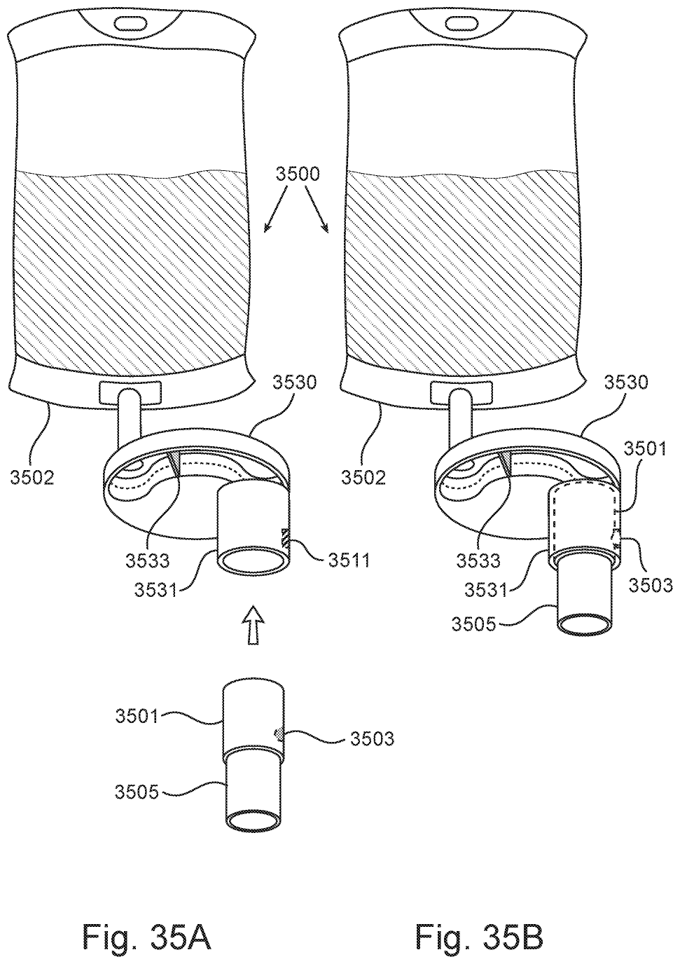

FIGS. 33A-33B illustrate a system 3300 which includes a decontamination device 3330 and a first vessel 3301 configured to engage with decontamination device 3330 via port 3331. Decontamination device 3330 houses a wiping member (not shown) and vessel 3301 is configured to move via rotational movements within the device 3330 and thereby undergo wiping/decontamination of a surface of vessel 3301. An end point of a rotational movement of vessel 3301 includes a longitudinal alignment with vessel 3302 and thereafter fluid transfer between the vessels. Vessel 3301 includes a lock mechanism 3303 in the form of a retractable ball which may be magnetic. Lock mechanism 3303 of vessel 3301 may optionally be a pin and/or a latch locking mechanism similar and/or identical to pin and/or latch locking mechanisms previously described in this specification. An unlock mechanism such as magnetic dent 3311 may be disposed on port 3331 and may attract ball 3303 when vessel 3301 engages port 3331. Unlock mechanism 3311 may optionally be a pin, a pin and spring mechanism, or any of the unlock mechanisms previously described in this specification. A similar system 3400 is shown in FIGS. 34A-34E which illustrate decontamination device 3430 that accommodates wiping member 3433 and that can be engaged with syringe 3401 via port 3431. The lock mechanism may include a retractable ball 3403, which may be magnetic. The unlocking mechanism may be a dent/magnet 3411. The engagement between vessel 3401 and port 3431 effects movement/withdrawal of ball 3403 into dent 3411 (FIGS. 34B and 34D). Vessel 3401 may be moveable within device 3430 via rail 3441 such that upon the movement of the vessel 3401 a surface thereof is wiped off and decontaminated by wiping member 3433. The movement of vessel 3401 within device 3430 allows wiping off a surface of the vessel 3401. The vessel 3401 moves/rotates in the device 3430 until reaching a longitudinal alignment with vessel 3402 such to allow a fluid passageway between vessel 3401 and 3402 (FIG. 34E). A similar system 3500 is shown in FIGS. 35A-35B which illustrate yet another form of first vessel being cartridge 3501 finger protective side walls 3505. Decontamination device 3530 accommodates wiping member 3533 and can be engaged with syringe 3501 via port 3531. The lock mechanism includes a retractable ball 3503, which may be magnetic. The unlocking mechanism may be a dent/magnet 3511. A plunger 3505 has a form suitable for accommodating a finger of a user, thus effecting an injection of a fluid within vessel 3501. Lock mechanism 3503 of cartridge 3501 may optionally, or alternately, be a pin and/or latch lock mechanism configured to be unlocked by unlocking mechanism 3511 which may be a pin and/or pin with spring unlocking mechanism.

Each of the following terms: ‘includes’, ‘including’, ‘has’, ‘having’, ‘comprises’, and ‘comprising’, and, their linguistic, as used herein, means ‘including, but not limited to’, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase ‘consisting essentially of’.

Each of the phrases ‘consisting of’ and ‘consists of’, as used herein, means ‘including and limited to’.

The term ‘method’, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Throughout this disclosure, a numerical value of a parameter, feature, characteristic, object, or dimension, may be stated or described in terms of a numerical range format. Such a numerical range format, as used herein, illustrates implementation of some exemplary embodiments of the invention, and does not inflexibly limit the scope of the exemplary embodiments of the invention. Accordingly, a stated or described numerical range also refers to, and encompasses, all possible sub-ranges and individual numerical values (where a numerical value may be expressed as a whole, integral, or fractional number) within that stated or described numerical range. For example, a stated or described numerical range ‘from 1 to 6’ also refers to, and encompasses, all possible sub-ranges, such as ‘from 1 to 3’, ‘from 1 to 4’, ‘from 1 to 5’, ‘from 2 to 4’, ‘from 2 to 6’, ‘from 3 to 6’, etc., and individual numerical values, such as ‘1’, ‘1.3’, ‘2’, ‘2.8’, ‘3’, ‘3.5’, ‘4’, ‘4.6’, ‘5’, ‘5.2’, and ‘6’, within the stated or described numerical range of ‘from 1 to 6’. This applies regardless of the numerical breadth, extent, or size, of the stated or described numerical range.

Moreover, for stating or describing a numerical range, the phrase ‘in a range of between about a first numerical value and about a second numerical value’, is considered equivalent to, and meaning the same as, the phrase ‘in a range of from about a first numerical value to about a second numerical value’, and, thus, the two equivalently meaning phrases may be used interchangeably.

The term ‘about’, is some embodiments, refers to ±30% of the stated numerical value. In further embodiments, the term refers to ±20% of the stated numerical value. In yet further embodiments, the term refers to ±10% of the stated numerical value.

It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub combination in the context or format of a single embodiment, may also be illustratively described and presented in the context or format of a plurality of separate embodiments.

Although the invention has been described in conjunction with specific embodiments thereof, it is evident that many alternatives, modifications, and variations will be apparent to those skilled in the art. Accordingly, it is intended to embrace all such alternatives, modifications, and variations that fall within the spirit and broad scope of the appended claims.

All publications, patents, and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A system for unlocking a plunger of a vessel and for the engagement and establishment of fluid passageway between vessels, the system comprising:
a first vessel;
a second vessel configured to be coupled to the first vessel; and
a plunger lock mechanism disposed on the first vessel and configured to lock a plunger thereof and prevent the plunger from expelling content of the first vessel prior to an engagement of the first vessel with the second vessel, wherein the first and/or second vessel(s) comprise(s) an unlock mechanism configured to unlock the lock mechanism of the first vessel, and wherein at the time of or following the engagement between the first and second vessels, the unlock mechanism unlocks the lock mechanism of the plunger, allowing movement thereof and thereby a fluid transfer from the first vessel into the second vessel.

2. The system of claim 1, wherein the lock mechanism is selected from a group consisting of a latch, a spring, a magnet, a pin, a retractable pin, a ball, a retractable ball, and a combination thereof.

3. The system of claim 1, wherein the unlock mechanism is selected from a group consisting of a latch, a spring, a magnet, a hook, a tooth, a pin, a retractable pin, a knob, a retractable knob, a ball, a retractable ball, a thread, a bangle, a locking key, a dent, a socket, a channel, an elongated arm, a latch, a lock, a slot, and a combination thereof.

4. The system of claim 1, wherein the lock mechanism is moveable between a locked position wherein the lock mechanism protrudes from a side wall of the first vessel such to disrupt movement forward of the plunger and an unlocked retracted position wherein the plunger is capable of moving forward thereby expelling content of the first vessel.

5. The system of claim 1, wherein the lock and/or unlock mechanism is pivotally connected to a side wall of the first vessel and moveable about said pivot.

6. The system of claim 1, wherein the lock and/or unlock mechanism is spring loaded or attached to a spring, allowing planar movement thereof.

7. The system of claim 1, wherein the lock mechanism includes a retractable ball which can protrude into an inner lumen of the vessel thereby lock the plunger and retract to a socket in a side wall of the first and/or second vessel, thereby unlock the plunger.

8. The system of claim 1, wherein the lock mechanism includes a magnetic ball which can protrude into an inner lumen of the vessel thereby lock the plunger and retract to a magnetic socket in a side wall of the first and/or second vessel, thereby unlock the plunger.

9. The system of claim 1, wherein the unlock mechanism includes an elongated arm disposed in the second vessel and configured to unlock the lock mechanisms.

10. The system of claim 1, further comprising an engagement mechanism to connect the first vessel to the second vessel.

11. The system of claim 10, wherein the engagement mechanism is selected from a thread, a luer, a ratchet teeth mechanism, a retention member, an adhesive mechanism, a clamping member and combinations thereof.

12. The system of claim 1, wherein at least one of the first vessel and second vessel is selected from the group consisting of a bag, a bottle, a syringe, an infusion line, a connector, a filter, a manifold, a bag port, a bottle port, a vial port, a cartridge and a combination thereof.

13. The system of claim 1, wherein the first and/or second vessel includes a port having peripheral walls enclosing a cavity, the cavity configured to receive or engage with a vessel, or a portion thereof.

14. The system of claim 12, wherein the first vessel is a cartridge.

15. The system of claim 14, wherein the second vessel includes a port having peripheral walls enclosing a cavity, the cavity is sized and shaped to accommodate the cartridge.

16. The system of claim 1, further comprising a decontamination device configured to be coupled to the first vessel and second vessel and allow the fluid passageway therebetween, the decontamination device further configured to decontaminate a surface of at least one of the first and second vessels.

17. The system of claim 16, further comprising an engagement mechanism to connect the decontamination device to the first and/or second vessel.

18. The system of claim 16, wherein the vessel and/or decontamination device includes a port having peripheral walls enclosing a cavity, the cavity configured to receive or engage with a vessel or a portion thereof.

19. The system of claim 16, wherein the decontamination device is pre-attached to the first or second vessels.

20. The system of claim 19, wherein the decontamination device is integrally attached to the first or second vessel.

21. The system of claim 16, wherein the decontamination device includes a wiping member configured to wipe off a surface of a vessel.

22. The system of claim 21, wherein at least a portion of the first and/or second vessel is configured to move within the device and wherein the wiping member configured to remove contaminants from a surface of said first and/or second vessels at about the time of said vessel's movement.

23. The system of claim 21, wherein the wiping member is configured to move within the device such to allow decontamination of a surface of at least one of said first and second vessels at about the time of said wiping member's movement.

24. The system of claim 21, wherein the decontamination device includes a sliding mechanism located on and/or attached to said wiping member and/or said vessel and configured to allow for the movement of said wiping member and/or said vessel within said device.

25. The system of claim 16, wherein the lock mechanism disposed on the first vessel and the unlock mechanism disposed on the decontamination device and/or first vessel.

26. The system of claim 1, wherein the unlock mechanism includes an elongated arm disposed on the second vessel and configured to unlock the lock mechanisms.
